(19) United States
(12) Patent Application Publication
(10) Pub. No.: US 2024/0358783 A1
(43) Pub. Date: Oct. 31, 2024

(54) METHOD AND SYSTEM FOR THE TRANSDERMAL ADMINISTRATION OF A PSYCHEDELIC AGENT

(71) Applicant: LYFE CHNG, LLC, South Lake Tahoe, CA (US)

(72) Inventors: Frank KOCHINKE, San Jose, CA (US); Chad CONNER, San Diego, CA (US); Bryan ARRAYALES, Los Angeles, CA (US)

(21) Appl. No.: 18/767,841

(22) Filed: Jul. 9, 2024

Related U.S. Application Data
(63) Continuation of application No. 17/710,972, filed on Mar. 31, 2022.
(60) Provisional application No. 63/169,171, filed on Mar. 31, 2021.

(51) Int. Cl. A61K 36/185; A61K 9/70; A61K 31/375; A61K 31/404; A61K 36/06

(52) U.S. Cl. CPC A61K 36/185; A61K 9/7038; A61K 31/375; A61K 31/404; A61K 36/06

(57) ABSTRACT

A method and drug delivery system are provided for transdermally administering a psychedelic active agent to a subject to provide continuous microdose plasma levels of the active agent or a metabolite thereof during an extended drug delivery time period. The transdermal drug delivery system comprises a drug reservoir that houses a formulation containing the active agent, a combination of a solubilizer-type permeation enhancer and a plasticizer-type permeation enhancer, and a pH stabilizing agent. The pH stabilizing agent brings the pH of the formulation, at the system-skin interface, and/or within the skin as the active agent is transported across the skin from the skin surface to the bloodstream, to within 25% of the pKa of the active agent. Formulations and methods of use are also provided.

### CROSS-REFERENCE TO RELATED APPLICATION

This application is a continuation of U.S. patent application Ser. No. 17/710,972, filed Mar. 31, 2022, which claims priority under 35 U.S.C. § 102 (e) (1) to provisional U.S. Patent Application No. 63/169,171, filed Mar. 31, 2021, the disclosures of which are hereby incorporated by reference.

### TECHNICAL FIELD

The present invention relates generally to the use of psychedelic agents in pharmacotherapy, and, more particularly, relates to the transdermal administration of psychedelic agents for the prevention and treatment of various disorders, diseases, and other adverse conditions.

### BACKGROUND

In recent years, research into potential therapeutic uses of psychedelic agents, primarily those extracted from mushrooms, has been accelerated. Many of the naturally occurring psychedelic agents are indole-based, e.g., tryptamines, including 5-hydroxytryptamine (serotonin) and derivatives thereof. The indole and tryptamine base structures are shown below along with the standard numbering system used to indicate the substitutable positions on the molecular structures:

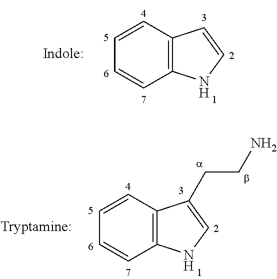 [SMILES: c1ccc2[nH]ccc2c1 ; NCCc1c[nH]c2ccccc12]

Of the about 600 naturally occurring indole compounds, psilocybin has been one of the most researched, and has been identified as an attractive and potentially powerful agent in the treatment and management of numerous mental health conditions. Psilocybin has the molecular structure

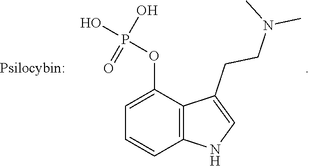 [SMILES: CN(C)CCc1c[nH]c2cccc(OP(=O)(O)O)c12]

The polarity of the phosphate group combined with the presence of the dimethylamino functionality makes the molecule zwitterionic in the environment and at physiological pH.

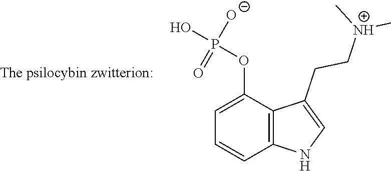 [SMILES: C[NH+](C)CCc1c[nH]c2cccc(OP(=O)([O-])O)c12]

Psilocybin can be found in more than 200 species of Psilocybe mushrooms, including P. azurescens, P. cyanescens, P. cubensis, and P. semilanceata. While taxonomically these species are all considered members of their respective species (and most are P. cubensis), they can differ drastically in appearance, conditions required for cultivation, and potency. See, e.g., Stamets, Psilocybin Mushrooms of the World (Ten Speed Press, 1995). These mushrooms contain many different psychoactive agents, mostly tryptamines, with psilocybin typically having the highest concentration. The levels and ratios of other psychedelic compounds, mostly psilocin and baeocystin, vary from species to species. The rare P. azurescens is the most potent of the Psilocybe mushrooms, as it contains the highest concentrations of psilocybin and psilocin, while P. cubensis is the species that is commonly referred to as “magic mushrooms.”

The pharmacokinetics, pharmacology and human metabolism of psilocybin are well known and well characterized. See, e.g., Bauer, “The Pharmacology of Psilocybin and Psilocin,” Psychedelic Science Review (Mar. 13, 2019); Passie et al. (2002), “The pharmacology of psilocybin,” Addiction Biology 7 (4): 357-64; Brown et al. (2017), “Pharmacokinetics of Escalating Doses of Oral Psilocybin in Healthy Adults,” Clin. Pharmacokinet. 56 (12): 1543-54; and Dinis-Oliveira (2017), “Metabolism of psilocybin and psilocin: clinical and forensic toxicological relevance,” Drub Metab. Rev. 49 (1): 84-91. Psilocybin has been utilized broadly in Phase 2 clinical trials conducted in the academic setting. The profound impact of psilocybin and other agents derived from Psilocybe mushrooms include changes in sensory perception, emotion, thought, and sense of self, characterized by marked alterations in many, if not all, mental functions, including perception, mood, volition, cognition, and self-experience. In early clinical studies, preliminary efficacy was established in the treatment of obsessive compulsive disorder (OCD), substance use disorder, depression, and anxiety.

The clinical safety of psilocybin has been extensively studied, both as a single agent and as adjunctive treatment in adult populations. Psilocybin has been administered via oral and/or intravenous delivery and studied in both open-label and double-blind controlled trials; see the Usona Institute Investigator's Brochure for IND #129532 (Dec. 17, 2018). In the aforementioned study, oral dosages ranged from 0.014 mg/kg to 0.6 mg/kg (about 1 mg to 40 mg for the average person), administered as either a single dose or divided between multiple doses spaced apart. It was shown that the effects of psilocybin are dose-dependent, although other factors such as personality structure and setting appear to modulate its overall effects. Although most test subjects experience profound changes in mood, perception, thought, and self-experience, at higher doses acute adverse drug reactions, characterized by strong dysphoria and/or anxiety/panic, can occur.

Although many researchers have exhibited interest in the potential pharmaceutical applications of psilocybin, there are numerous obstacles to the successful implementation of psilocybin as a pharmaceutical agent. For example, when psilocybin is administered to a patient orally or intravenously, significant physiological and psychological adverse events can result. Oral administration of Psilocybe mushrooms, for example, can cause severe gastrointestinal distress; see Bauer (2019), supra. Regarding psychological side effects, psilocybin is well known to result in paranoia, panic, and psychosis in some individuals and at higher doses. There are several other safety-related issues as well.

Additionally, when psilocybin is orally or intravenously administered, it must be converted into its active metabolite, the 4-hydroxy analog of psilocybin—psilocin—in order to achieve the desired therapeutic effect in the brain; the zwitterionic psilocybin is very water soluble and unable to pass through the blood-brain barrier or other physiological membranes. In contrast to psilocybin, psilocin can cross the blood-brain barrier. It has been confirmed that the CNS effects of psilocybin are seen only after transformation to psilocin (see, e.g., Horita and Weber, “Dephosphorylation of psilocybin in the intact mouse,” Toxicology and Applied Pharmacology 4 (6): 730-737). Psilocybin is thus a prodrug for the active form of the molecule, psilocin.

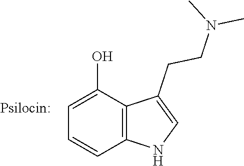

Alkaline phosphatase (ALP) is the primary enzyme that dephosphorylates psilocybin, a rapid reaction that takes place in the intestinal mucosa. Other phosphatases dephosphorylate psilocybin in the intestines, kidneys, and blood. However, ALP concentrations vary drastically from patient to patient as a function of age, underlying medical conditions, and/or ongoing medical treatments (e.g., chemotherapy). Due to the variability of ALP concentrations, the resulting concentration of psilocin will vary too. Consequently, administration of psilocybin as attempted by others has resulted in unpredictable and inconsistent therapeutic effects as well as potentially dangerous side effects as noted above.

Although some researchers have suggested that oral administration of psilocybin is safe, users have complained about undesirable systemic reactions and other adverse events, as noted previously; the most critical adverse event is the undesirable “bad trip” experience, i.e., an unpleasant psychotropic effect. This can happen if the dosage and/or patient ALP concentration is too high. Another not often discussed serious pathological condition is Serotonin (5-HT) Syndrome (SS, serotonin toxicity or hyperserotonemia), resulting from excess serotonergic activity in the brain. SS is one of the most important neurotoxic syndromes, and commonly develops due to an overdose of serotonergic antidepressants or interactions between several serotonergic drugs. Depending on the degree of toxicity, SS manifests clinically as a characteristic triad of neuromuscular, autonomic and mental symptoms, which range from mild to life-threatening, and can develop rapidly, within hours after drug administration. Early symptoms of SS typically include tachycardia, shivering, diaphoresis, mydriasis, tremor and hyperreflexia. Progression from mild to moderate serotonin toxicity produces hypertension, fever, and mental status changes (such as confusion, hypomania, anxiety and agitation). Finally, severe SS cases involve muscular rigidity, hypertonicity, hyperthermia, seizures and coma.

An alternative, non-oral route of administration might be considered to address the foregoing limitations, with the transdermal route of particular interest insofar as continuous administration of a low dose of the drug would obviate the known adverse effects. The GI system would be bypassed, such that GI side effects would be avoided, and the psychological and neurological side effects associated with higher doses and/or higher patient ALP concentrations would be avoided as well. However, psilocybin is unable to pass through the skin. Furthermore, the active metabolite, psilocin, would also be presumed to be a poor candidate for transdermal administration because it is a polar molecule bearing localized charges on the hydroxyl and amino functionalities. It is well known in the art of transdermal drug delivery that charged molecules-which may be zwitterions, cations or anions associated with a counterion, or polar molecules with localized charge-tend not to pass through the skin. Charged molecules, therefore, are generally not considered candidates that for transdermal administration. See, e.g., Tanwar et al. (2016), “Transdermal Drug Delivery: A Review,” Int. J. Pharm Sci Res 7 (6): 2274-90.

Accordingly, there is a need in the art for a way to enable the administration of psychedelic agents such as psilocin before such compounds can be safely and effectively administered to patients in the therapeutic context. The present invention is addressed to the aforementioned need in the art.

### SUMMARY OF THE INVENTION

Psilocybin, a zwitterion, is unable to cross the blood brain barrier to achieve a therapeutic effect in the brain, as explained above. The same holds true for the potential transdermal administration of psilocybin, insofar as charged drugs have very little or no skin permeability. It would be similarly presumed that psilocin, having a small, localized charge on the 4-hydroxyl group and the typically observed electron shift on the nitrogen atom, would not be a candidate for transdermal drug delivery. Surprisingly, however, it has now been found that psilocin is not only a viable candidate for transdermal drug delivery, but can, in fact, pass through the stratum corneum and upper layers of the skin and therefore be transdermally administered in to achieve a desired therapeutic effect.

The transdermal delivery of psilocin and other psychedelic agents as described and claimed herein allows for controlled blood concentration levels, in turn providing a therapeutic effect while avoiding “peaks and valleys” in concentration profile, where the high “peaks” may be associated with a “bad trip” experience and the “valleys” represent no therapy at all. In addition, if therapy is indicated to be stopped as a result of undesirable side effects, a transdermal system can be removed so that delivery immediately ceases, while an oral product cannot be removed, once administered, or an injection undone. Transdermal drug delivery as provided herein also avoids unpredictable effects resulting from variable alkaline phosphatase levels, as the gastrointestinal tract is bypassed. This is particularly important in the treatment of children or the elderly, who may be severely affected by high drug concentrations.

The invention provides a system, method, and formulation for the transdermal administration of psilocin and other psychedelic agents, avoiding the undesirable side effects when administered via the oral or intravenous route, insofar as transdermal administration as provided herein achieves continuous delivery of sub-threshold doses of a psychedelic agent, also referred to in the art as “microdoses.” By providing psychedelic microdosing throughout a transdermal drug delivery period, the invention has utility in any method in which psychedelic microdosing provides a desired therapeutic or other effect, such as improving creativity, boosting energy level, stabilizing mood, and treating anxiety, depression, addiction, and other conditions.

The psychedelic agents include, but not are limited to, serotonergic psychedelic agents, e.g., indolealkylamine compounds such as psilocin.

In one embodiment, then, the invention provides a drug delivery system for transdermally administering a psychedelic active agent to a subject through a localized region of the subject's skin. The system has an outer surface and a basal surface and comprises:
(a) a drug reservoir housing a formulation that comprises
   (i) about 0.5 wt. % to about 40 wt. % of the psychedelic active agent, the active agent having a known pKa,
   (ii) an effective skin permeation-enhancing amount of an enhancer composition comprising a combination of a solvent-type enhancer and a lipid disrupting enhancer, and
   (iii) an effective pH-adjusting amount of a pH-adjusting agent that maintains pH within the localized region to within 25% of the pKa; and
(b) a backing layer that may or may not be substantially impermeable to the formulation and serves as the outer surface of the system during drug administration.

The system also comprises a means for affixing the system to the skin, wherein the backing layer may be a skin contact adhesive that serves that purpose or an additional element, such as a layer (e.g., a rim) of a skin contact adhesive, is laminated to the drug reservoir and serves to affix the system to the subject's skin during drug administration.

In one aspect of the embodiment, the psychedelic active agent is a polar and/or ionizable molecule and the pH-adjusting agent is selected to maintain the pH within the localized region to within 15% of the pKa, and in some embodiments maintains the pH so as to approximate the pKa of the active agent. This may be in the drug reservoir, if the formulation contains a liquid vehicle that is at least partially aqueous, or it may be within the water-containing layers of the skin through which the active agent is transported to the bloodstream, or both.

In another aspect, the transdermal drug delivery system comprises a single layer adhesive system, wherein the drug reservoir is composed of a skin contact adhesive that serves to affix the system to the skin in addition to housing the active agent formulation. The system includes a removable release liner that protects the basal surface of the system during storage and prior to drug delivery.

In an additional aspect, the transdermal drug delivery system comprises a monolithic matrix system in which the drug reservoir again comprises a polymeric matrix but it does not also serve as the means for affixing the system to the skin. Rather, a separate skin contact adhesive layer is laminated to the skin side of the drug reservoir.

In another aspect, the drug reservoir comprises an enclosed pouch that houses the active agent-containing formulation, such that the system is a liquid reservoir system. In one design, the reservoir is located between an outer backing layer and a heat-sealed membrane, wherein the heat-sealed membrane is laminated to a skin contact adhesive layer for affixing the system to the skin. The membrane may be selected to control the rate of drug release from the system, the rate of enhancer release from the system, or both. In another design, a microporous, non-adhesive reservoir is substituted for the pouch in the aforementioned embodiment, with the reservoir directly contacting the skin. A skin contact adhesive layer is incorporated between the outer backing layer and the microporous reservoir. In order to facilitate adhesion to the skin, the surface area of the microporous reservoir is smaller than the surface area of the skin contact adhesive layer, such that the skin contact adhesive layer extends beyond the periphery of the reservoir, with the uncovered, i.e., exposed, adhesive area available to affix the system to the skin.

In another aspect, the formulation in the drug reservoir additionally comprises at least one excipient selected from viscosity adjusting agents, emulsifiers, solubilizers, preservatives, opacifiers, colorants, fragrance, and irritation-mitigating additives. An excipient of particular interest is a formulation stabilizer, which may be an active agent stabilizer such as ascorbic acid or an ascorbate salt.

In a further aspect, the transdermal delivery system provides for sustained release of the active agent throughout an extended drug delivery period in the range of about 6 hours to about 84 hours, e.g., in the range of about 8 hours to about 24 hours.

In still a further aspect the psychedelic agent is present in an amount effective to provide a blood level in the range of 0.5 ng/ml to about 5.0 ng/ml during the extended drug delivery time period.

In another embodiment, a drug delivery system is provided for transdermally administering psilocin to a subject a localized region of the subject's skin, the system having an outer surface and a basal surface and comprising:
(a) a drug reservoir housing a formulation that comprises
   (i) about 0.5 wt. % to about 40 wt. % psilocin,
   (ii) an amount of a buffering agent that adjusts maintains pH in the range of 7.20 to 9.74 within the localized region; and
   (iii) 0.5 wt. % to 15 wt. % of a monoamine oxidase inhibitor; and
(b) a backing layer that serves as the outer surface of the system during drug administration, wherein either
(c) the drug reservoir is comprised of a skin contact adhesive that serves as a means for affixing the system to the subject's skin, or
(d) a layer of a skin contact adhesive material is laminated to the drug reservoir and serves as a means for affixing the system to the subject's skin.

In one aspect of the embodiment, the formulation additionally includes an active agent stabilizer comprises an oxygen scavenger, such that the psilocin is protected from oxygen-induced degradation and/or oxygen-induced reactions during storage and prior to use. The active agent stabilizer may be ascorbic acid or an ascorbate salt, and typically represents about 0.5 wt. % to about 15 wt. % of the formulation.

In another aspect of the embodiment, the pH-adjusting agent is selected to change the pH in the localized region of the active agent so that it approximates the pKa of psilocin, which is 8.47. The localized region of the psilocin may be within the drug reservoir, if the psilocin formulation includes a liquid vehicle that is at least partially aqueous, or it may be within the water-containing layers of the skin after psilocin is released into the skin, or both.

In a further embodiment, the invention provides a drug delivery system for transdermally administering psilocin to a subject through a localized region of the subject's skin, the system having an outer surface and a basal surface and comprising:
(a) a drug reservoir housing a formulation that comprises
   (i) 2.5 wt. % to 25 wt. % psilocin,
   (ii) 1.0 wt. % to 15 wt. % sodium bicarbonate,
   (iii) 1.0 wt. % to 15 wt. % ascorbic acid, and
   (iv) 1.0 wt. % to 15 wt. % of a monoamine oxidase inhibitor; and
(b) a backing layer that serves as the outer surface of the system during drug administration, wherein either
(c) the drug reservoir is comprised of a skin contact adhesive that serves as a means for affixing the system to the subject's skin, or
(d) a layer of a skin contact adhesive material is laminated to the drug reservoir and serves as a means for affixing the system to the subject's skin.

In aspects of the aforementioned embodiments, the transdermal system used to administer the psychedelic agent to a subject provides a total flux J_{T} of active agent released from the system and across the skin that is primarily determined by the system flux, or patch flux, J_{P}.

In other aspects, the transdermal delivery system releases the active agent at a patch flux J_{P}, the active agent is transported across the skin at a skin flux J_{S}, and the ratio J_{S}: J_{P} is in the range of about 2:1 to about 100:1.

In another embodiment, a modular transdermal drug delivery system is used to deliver the psychedelic active agent in a formulation as before, wherein the components of the system are analogous to those described in U.S. Patent Publication No. 2020/0268681 A1 to Kochinke, incorporated herein by reference.

In another embodiment, the invention provides a method for administering a psychedelic agent to a subject by affixing any of the above-mentioned transdermal systems of formulations to a localized region of the subject's skin and allowing the system to remain in place throughout an extended drug delivery time period.

In one aspect of the embodiment, the psychedelic active agent is administered to a subject at continuous microdosing levels.
psychedelic active agent to a subject at continuous microdosing levels using a transdermal system of the invention.

In a further embodiment, the invention provides a method for administering a psychedelic active agent to a subject at continuous microdosing levels using a formulation of the invention.

In an additional embodiment, the method provides a method for administering a psychedelic agent to a subject provide to provide a desired beneficial effect, by transdermally administering the psychedelic agent using a transdermal system of the invention.

In another embodiment, the method provides a method for administering a psychedelic agent to a subject provide to provide a desired beneficial effect, by transdermally administering the psychedelic agent using a formulation of the invention.

In aspects of the aforementioned embodiments, the desired beneficial effect is a therapeutic effect.

In another embodiment, a method is provided for administering a psychedelic agent to a subject to increase neocortical 5-HT_{2A} receptor occupancy to a desired level, the method comprising:

transdermally administering a psychedelic agent to the subject in a manner effective to provide, throughout a drug delivery time period, a plasma level of the agent or metabolite thereof that corresponds to the desired level of 5-HT_{2A} receptor occupancy.

In one aspect of the embodiment, the psychedelic agent is administered to the subject by affixing a transdermal drug delivery system to the subject's skin, wherein the drug delivery system releases the active agent at a patch flux J_{P}, the active agent is transported across the skin at a skin flux J_{S}, and the ratio J_{S}: J_{P} is in the range of about 2:1 to about 100:1.

In another aspect of the embodiment, the psychedelic agent is administered to the subject by affixing a transdermal drug delivery system to the subject's skin to provide a total flux J_{T} that is primarily determined by patch flux J_{P} of the agent being released by the system, rather than by skin flux J_{S} of the agent across the skin,
wherein the system comprises an effective amount of the psychedelic agent;
an effective skin permeation-enhancing amount of a combination of a solvent-type enhancer and a lipid-disrupting enhancer; and a pH adjusting agent.

Additional objects, advantages, and salient features of exemplary embodiments of the invention will become apparent to those skilled in the art from the following description.

### BRIEF DESCRIPTION OF THE DRAWINGS

FIG. 1 schematically illustrates the plasma concentration profile of an active agent or metabolite thereof delivered via macrodose delivery versus that seen with microdose delivery.

FIG. 2 schematically illustrates a simple adhesive system for transdermally administering an active agent according to the invention.

FIG. 3 schematically illustrates a monolithic matrix system for transdermally administering an active agent according to the invention.

FIG. 4 schematically illustrates a liquid reservoir system for transdermally administering an active agent according to the invention, where the liquid reservoir comprises a cavity enclosed by a heat-sealed membrane and an occlusive outer backing layer.

FIG. 5 schematically illustrates a liquid reservoir system for transdermally administering an active agent according to the invention, where a microporous reservoir is loaded with the active agent-containing formulation to be delivered.

FIG. 6 is a graph showing the cumulative mass released over time, from the transdermal psilocin systems prepared in Examples 2 and 3.

FIG. 7 is a graph showing the skin flux over time obtained with the transdermal psilocin systems prepared in Examples 2 and 3.

### DETAILED DESCRIPTION OF THE INVENTION

### 1. Definitions and Terminology

Unless defined otherwise, all technical and scientific terms used herein have the meaning commonly understood by one of ordinary skill in the art to which the invention pertains. Specific terminology of particular importance to the description of the present invention is defined below.

In this specification and the appended claims, the singular forms “a,” “an” and “the” include plural referents unless the context clearly dictates otherwise. Thus, for example, reference to “a pharmacologically active agent” or simply “an active agent” includes a single such agent as well as two or more such agents; “a pharmaceutically acceptable carrier” refers to a combination of pharmaceutically acceptable carriers as well as to a single pharmaceutically acceptable carrier; “a formulation” and “a vehicle” includes two or more formulations and vehicles, respectively, and the like.

The term “active agent” refers to any chemical compound, complex or composition that exhibits a desirable pharmacological, physiological, psychoactive, or other beneficial effect. For instance, in certain embodiments, the active agent may be a pharmacologically active agent that exerts a therapeutic effect in the treatment of an adverse physiological condition. The term also encompasses an agent that interacts with a body, or a portion thereof, to produce a desired condition, for example. It is also to be understood that in certain embodiments, an active agent need not be a pharmacologically active agent nor need it have a pharmaceutical effect so long as the effect it does have is deemed beneficial by the subject to whom the agent is administered.

When referring to an active agent, whether specified as a particular compound (e.g., psilocin) or a compound class (e.g., psychedelic agent), the term used to refer to the agent is intended to encompass not only the specified molecular entity but also its pharmaceutically acceptable, pharmacologically active analogs and derivatives, including, but not limited to, salts, esters, amides, prodrugs, conjugates, active metabolites, hydrates, crystalline forms, enantiomers, stereoisomers, and other such derivatives, analogs, and related compounds.

A “psychedelic” active agent as that term is used herein refers to a pharmacologically active agent that tends to bring about modifications in a subject's perception, emotion, cognition, and volition, and which can, at high doses, cause hallucinogenic effects. With the present invention, hallucinogenic effects and other adverse effects are avoided without compromising therapeutic efficacy. The psychedelic active agents administered herein raise brain entropy within or slightly above the critical zone defined by Carhart-Harris (2018), “The Entropic Brain-Revisited,” Neuropharmacology 142:167-178.

By “transdermal” delivery, applicants intend to include both transdermal (or “percutaneous”) and transmucosal administration, i.e., delivery by passage of a pharmacologically active agent through the skin or mucosal tissue and into the bloodstream, thereby providing a systemic effect. “Topical” delivery generally refers to delivery of an active agent to the skin surface and thus the uppermost region of the skin, and provides a local rather than systemic effect, as the agent does not penetrate into the bloodstream. The present systems are applied “topically” insofar as they are affixed to a body surface, but depending on the active agent and the components of the formulation containing the active agent (such as one or more permeation enhancers), drug delivery may be either transdermal or topical and thus either systemic or local, although systemic drug delivery is generally preferred herein. Accordingly, unless otherwise specified herein, reference to a “transdermal system” encompasses a system that can be used for either transdermal delivery or topical delivery, and reference to “transdermal delivery” encompasses a method that can be adapted for either transdermal or topical drug administration.

The terms “treating,” “treatment,” and “therapeutic” as used herein refer to the administration of a pharmaceutical agent or composition to a subject to provide a desired pharmacological or physiological effect, and thus encompasses administration for therapeutic and/or prophylactic purposes. Treating a condition in a subject already suffering from that condition generally involves a reduction in the severity, number, and/or frequency of symptoms, the elimination of symptoms and/or underlying cause, and the improvement or remediation of damage. In the prophylactic context, treatment refers to the administration of a pharmaceutical agent or composition to a subject who is not yet suffering from a particular condition, but has been identified as at susceptible to, i.e., at risk for developing, the particular condition, where the prophylactic effect involves partially or completely preventing a condition or symptom thereof.

The terms “effective amount,” “therapeutically effective amount,” and “therapeutically effective concentration” of an active agent, an active agent combination, or a pharmaceutical formulation refer to an amount or concentration that is nontoxic but sufficient for producing a desired result. The exact amount required will vary from subject to subject, depending on factors such as the age, weight and general condition of the subject, the particular condition being treated, the severity of the condition, the specific active agent, and, of course, the judgment of the clinician.

The effective amount of a psychedelic active agent in the present systems and formulations is such that a “microdose” of the active agent is administered to a subject throughout an extended drug delivery time period. The term “microdose” herein, referring to the preferred dose of the psychedelic, agent, refers to a dose below a “macrodose,” i.e., a dose that results in a psychedelic experience, and is generally at most about 20%, or at most about 15%, or at most about 10%, of the macrodose. Generally, although not necessarily, a microdose is also below a threshold dose, meaning the lowest dose at which mental and/or physical alterations produced by the psychoactive agent are experienced by the subject. Depending on the particular active agent employed, a microdose may be less than 100 mg, less than 75 mg, less than 50 mg, less than 20 mg, less than 10 mg, less than 5 mg, less than 1 mg, less than 500 μg, less than 250 μg, less than 200 μg, less than 150 μg, less than 100 μg, less than 50 μg, 0 less than 25 μg, less than 20 μg, and so forth.

By “pharmaceutically acceptable” is meant a material that is not biologically or otherwise undesirable, i.e., the material may be incorporated into a pharmaceutical composition as provided herein and not cause any substantial undesirable biological effects or interact in a deleterious manner with any of the other components of the composition. When the term “pharmaceutically acceptable” is used to refer to a pharmaceutical carrier or excipient, it is implied that the carrier or excipient has met the required standards of toxicological and manufacturing testing and/or that it is included on the Inactive Ingredient Guide prepared by the U.S. Food and Drug Administration and designated “Generally Regarded as Safe” (“GRAS”).

As used herein, a “subject” or “individual” or “patient” refers to any subject for whom therapy is desired, and generally refers to the recipient of the therapy to be practiced according to the invention.

A “drug delivery time period” refers to a period of time during which the active agent is delivered from a transdermal system or formulation into the skin of a subject, and is generally an extended time period in the range of about 6 hours to about 84 hours, e.g., about 8 hours to about 24 hours.

“Optional” or “optionally present”—as in an “optional additive” or an “optionally present additive” means that the subsequently described component (e.g., additive) may or may not be present, so that the description includes instances where the component is present and instances where it is not.

The term “substantially” indicates the possibility of slight deviation from a recited chemical or physical property, and allows for a difference of at most about 20%, or at most about 10%, or at most about 5%, between an actual chemical or physical property and the recited chemical or physical property. The term “substantially homogeneous,” for example, refers to a material in the form of a mixture of two or more components in which the material is substantially uniform throughout, with any two discrete regions within the material differing by at most about 20%, or at most about 10%, or at most about 5%, with respect to a chemical or physical property of the material, such as the presence or absence of a component, particle size, the concentration of a component, the degree of hydrophilicity or lipophilicity, density, crystallinity, or the like.

Similarly, the terms “approximately” and “about” in any context is intended to connote a possible variation of at most about 20%. Generally, the term connotes a possible variation of at most about 10%, or at most about 5%.

### Chemical Substituent and Compound Terminology:

As used herein, the phrase “having the structure” is not intended to be limiting and is used in the same way that the term “comprising” is commonly used.

The term “alkyl” as used herein refers to a branched or unbranched saturated hydrocarbon group typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl, and the like. Generally, although again not necessarily, alkyl groups herein contain 1 to about 18 carbon atoms, preferably 1 to about 12 carbon atoms. The term “lower alkyl” intends an alkyl group of 1 to 6 carbon atoms. Preferred lower alkyl substituents contain 1 to 3 carbon atoms, and particularly preferred such substituents contain 1 or 2 carbon atoms (i.e., methyl and ethyl). “Substituted alkyl” refers to alkyl substituted with one or more substituent groups, and the terms “heteroatom-containing alkyl” and “heteroalkyl” refer to alkyl in which at least one carbon atom is replaced with a heteroatom, as described in further detail infra. If not otherwise indicated, the terms “alkyl” and “lower alkyl” include linear, branched, cyclic, unsubstituted, substituted, and/or heteroatom-containing alkyl or lower alkyl, respectively.

The term “aryl” as used herein, and unless otherwise specified, refers to an aromatic substituent containing a single aromatic ring or multiple aromatic rings that are fused together, directly linked, or indirectly linked (such that the different aromatic rings are bound to a common group such as a methylene or ethylene moiety). Preferred aryl groups contain 5 to 24 carbon atoms, and particularly preferred aryl groups contain 5 to 14 carbon atoms. Exemplary aryl groups contain one aromatic ring or two fused or linked aromatic rings, e.g., phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituent, in which at least one carbon atom is replaced with a heteroatom, as will be described in further detail infra. If not otherwise indicated, the term “aryl” includes unsubstituted, substituted, and/or heteroatom-containing aromatic substituents.

The term “cyclic” refers to alicyclic or aromatic substituents that may or may not be substituted and/or heteroatom containing, and that may be monocyclic, bicyclic, or polycyclic.

The term “alicyclic” is used in the conventional sense to refer to an aliphatic cyclic moiety, as opposed to an aromatic cyclic moiety, and may be monocyclic, bicyclic, or polycyclic.

The term “heteroatom-containing” as in a “heteroatom-containing alkyl group” (also termed a “heteroalkyl” group) or a “heteroatom-containing aryl group” (also termed a “heteroaryl” group) refers to a molecule, linkage or substituent in which one or more carbon atoms are replaced with an atom other than carbon, e.g., nitrogen, oxygen, sulfur, phosphorus, silicon, or selenium, typically nitrogen, selenium, oxygen or sulfur, preferably nitrogen or selenium, or both nitrogen and selenium. Similarly, the term “heteroalkyl” refers to an alkyl substituent that is heteroatom-containing, the term “heterocyclic” refers to a cyclic substituent that is heteroatom-containing, the terms “heteroaryl” and heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, pyrimidinyl, imidazolyl, 1,2,4-triazolyl, tetrazolyl, etc., and examples of heteroatom-containing alicyclic groups are pyrrolidino, morpholino, piperazino, piperidino, etc.

“Hydrocarbyl” refers to univalent hydrocarbyl radicals containing 1 to about 30 carbon atoms, preferably 1 to about 24 carbon atoms, more preferably 1 to about 18 carbon atoms, most preferably about 1 to 12 carbon atoms, including linear, branched, cyclic, saturated, and unsaturated species, such as alkyl groups, alkenyl groups, aryl groups, and the like. “Substituted hydrocarbyl” refers to hydrocarbyl substituted with one or more substituent groups, and the term “heteroatom-containing hydrocarbyl” refers to hydrocarbyl in which at least one carbon atom is replaced with a heteroatom. Unless otherwise indicated, the term “hydrocarbyl” is to be interpreted as including substituted and/or heteroatom-containing hydrocarbyl moieties.

By “substituted” as in “substituted alkyl,” “substituted aryl,” “substituted hydrocarbyl,” and the like, as alluded to in some of the aforementioned definitions, is meant that in the alkyl, aryl, or other moiety, at least one hydrogen atom bound to a carbon (or other) atom is replaced with one or more non-hydrogen substituents. Examples of such substituents include, without limitation, additional hydrocarbyl groups, e.g., C₁-C₂₄ hydrocarbyl, C₁-C₁₂ hydrocarbyl, C₁-C₈ hydrocarbyl, and C₁-C₆ hydrocarbyl; functional groups such as halo, hydroxyl, sulfhydryl, C₁-C₂₄ alkoxy, C₂-C₂₄ alkenyloxy, C₂-C₂₄ alkynyloxy, C₅-C₂₄ aryloxy, acyl (including C₂-C₂₄ alkylcarbonyl (—CO-alkyl) and C₆-C₂₄ arylcarbonyl (—CO-aryl)), acyloxy (—O-acyl), C₂-C₂₄ alkoxycarbonyl (—(CO)—O— alkyl), C₆-C₂₄ aryloxycarbonyl (—(CO)—O-aryl), halocarbonyl (—CO)—X where X is halo), C₂-C₂₄ alkylcarbonato (—O—(CO)—O-alkyl), C₆-C₂₄ arylcarbonato (—O—(CO)—O-aryl), carboxy (—COOH), carboxylato (—COO—), carbamoyl (—(CO)—NH2), mono-(C₁-C₂₄ alkyl)-substituted carbamoyl (—(CO)—NH (alkyl)), di-(C₁-C₂₄ alkyl)-substituted carbamoyl (—(CO)—N(C₁-C₂₄ alkyl)₂), mono-(C₆-C₂₄ aryl)-substituted carbamoyl (—(CO)—NH-aryl), di-(C₆-C₂₄ aryl)-substituted carbamoyl (—(CO)—N(aryl)₂), di-N-(alkyl), N—(C₆-C₂₄ aryl)-substituted carbamoyl, thiocarbamoyl (—(CS)—NH₂), carbamido (—NH—(CO)—NH₂), cyano (—C≡N), isocyano (—N⁺≡C⁻—), cyanato (—O—C≡N), isocyanato (—O—N+≡C₋—), isothiocyanato (—S—C≡N), azido (—N═N+≡N″), formyl (—(CO)—H), thioformyl (—(CS)—H), amino (—NH₂), mono-(alkyl)-substituted amino, di-(alkyl)-substituted amino, mono-(C₅-C₂₄ aryl)-substituted amino, di-(C₅-C₂₄ aryl)-substituted amino, C₂-C₂₄ alkylamido (—NH—(CO)-alkyl), C₆-C₂₄ arylamido (—NH—(CO)-aryl), imino (—CR═NH where R=hydrogen, alkyl, C₅-C₂₄ aryl, C₆-C₂₄ alkaryl, C₆-C₂₄ aralkyl, etc.), alkylimino (—CR═N(alkyl), where R=hydrogen, C₁-C₂₄ alkyl, C₅-C₂₄ aryl, C₆-C₂₄ alkaryl, C₆-C₂₄ aralkyl, etc.), arylimino (—CR≡N(aryl), where R=hydrogen, C₁-C₂₄ alkyl, C₅-C₂₄ aryl, C₆-C₂₄ alkaryl, C₆-C₂₄ aralkyl, etc.), nitro (—NO₂), nitroso (—NO), sulfo (—SO₂—OH), sulfonato (—SO₂—O—), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₄ arylsulfinyl (—(SO)-aryl), C₁-C₂₄ alkylsulfonyl (—SO₂-alkyl), C₅-C₂₄ arylsulfonyl (—SO₂-aryl), phosphono (—P(O)(OH)₂), phosphonato (—P(O)(O—)₂), phosphinato (—P(O)(O—)), phospho (—PO₂), and phosphino (—PH₂); and the hydrocarbyl moieties C₁-C₂₄ alkyl (preferably C₁-C₁₈ alkyl, more preferably C₁-C₁₂ alkyl, most preferably C₁-C₆ alkyl), C₂-C₂₄ alkenyl (preferably C₂-C₁₈ alkenyl, more preferably C₂-C₁₂ alkenyl, most preferably C₂-C₆ alkenyl), C₂-C₂₄ alkynyl (preferably C₂-C₁₈ alkynyl, more preferably C₂-C₁₂ alkynyl, most preferably C₂-C₆ alkynyl), C₅-C₂₄ aryl (preferably C₅-C₁₄ aryl), C₆-C₂₄ alkaryl (preferably C₆-C₁₈ alkaryl), and C₆-C₂₄ aralkyl (preferably C₆-C₁₈ aralkyl).

In addition, the aforementioned functional groups may, if a particular group permits, be further substituted with one or more additional functional groups or with one or more hydrocarbyl moieties such as those specifically enumerated above. Analogously, the above-mentioned hydrocarbyl moieties may be further substituted with one or more functional groups or additional hydrocarbyl moieties such as those specifically enumerated.

When the term “substituted” appears prior to a list of possible substituted groups, it is intended that the term apply to every member of that group. For example, the phrase “substituted alkyl, alkenyl, and aryl” is to be interpreted as “substituted alkyl, substituted alkenyl, and substituted aryl.” Analogously, when the term “heteroatom-containing” appears prior to a list of possible heteroatom-containing groups, it is intended that the term apply to every member of that group. For example, the phrase “heteroatom-containing alkyl, alkenyl, and aryl” is to be interpreted as “heteroatom-containing alkyl, substituted alkenyl, and substituted aryl.”

### 2. Overview

Transdermal administration of pharmacologically active agents provides a convenient route of administration for a variety of indications. Delivery of an active agent via the transdermal route allows for continuous input of a therapeutic agent into systemic circulation and eliminates pulsed dosing peaks, which are problematic for many drugs and particularly for psychedelic agents, as explained earlier herein.

The process of percutaneous absorption has been described as follows: “when a drug system is applied topically, the drug diffuses passively out of its carrier or vehicle and into the surface tissue of the skin specifically and most importantly the stratum corneum and the sebum filled pilosebaceous gland ducts. A net mass movement continues through the full thickness of the stratum corneum and ducts into the viable epidermis and dermal strata. A concentration gradient is thus established across the skin, which essentially terminates at the outer reaches of the skin's microcirculation in the dermal layer. The systemic circulation acts as a ‘sink’ for the drug and near zero concentration of the drug is maintained at the plane where drug reaches the capillaries and is diluted into the general system. Once the drug is in general circulation it distributes very rapidly, and gives reasonable rates of systemic metabolism and elimination, i.e., there is generally systemic build-up. Thus, relatively high epidermal concentrations of some drugs may be obtained by reason of the fact that the epidermis is without a direct blood supply and the concentration gradient from the outer surface to the microcirculation cuts directly through the epidermis.” Flynn et al., Eds., Modern Pharmacokinetics (Marcel Dekker, 1979) at p. 263.

In order to deliver therapeutic agents through the skin to achieve a systemic effect, morphological and physicochemical properties of the skin must be considered. Skin is highly impermeable to most molecules on the basis of molecular size, hydrophilicity, lipophilicity, and charge. Large molecules, for instance, are difficult to administer transdermally, and as noted earlier herein, polar and charged molecules are not considered optimal candidates for transdermal delivery because they do not pass through the skin.

For the aforementioned reasons, various techniques have been adopted for enhancing the passage of an active agent through the skin for systemic delivery. One approach that has been widely used, and is adopted here, is the use of chemical permeation enhancers to facilitate drug permeation across the skin by increasing drug partitioning into the barrier domain of the stratum corneum. Penetration enhancers have several mechanisms of action such as increasing disruption of the stratum corneum bilayers and intercellular lipids and increasing the hydration of the stratum corneum to enhance solubility of a particular agent. For polar and charged drugs, electrical enhancement techniques have been employed, including iontophoresis and electroporation; passive transdermal delivery with chemical enhancers has not yielded optimal results.

In one embodiment, the invention provides systems, formulations, and methods for transdermally administering a psychedelic agent, such as an ionizable, ionized, and/or polar molecule, in which the agent is combined with a pH-adjusting agent, a combination of two enhancers, a stabilizing agent, if necessary, and, optionally, a pharmaceutically acceptable liquid vehicle. Although many psychedelic agents have undesirable side effects when administered via the oral or intravenous route, they can be administered according to the invention with no significant adverse effects, insofar as transdermal administration as provided herein achieves continuous delivery of sub-threshold doses of a psychedelic agent, also referred to in the art as “microdoses.” In the transdermal context, achieving microdose delivery is accomplished by ensuring that the blood concentration of active agent is maintained below a predetermined upper limit, above which adverse effects might occur. This is primarily ensured with the quantity of drug incorporated into the system, i.e., by the degree of drug loading, and by selection of both system and formulation components. Ideally, the total input flux J_{T} of drug from the system, through the skin, and into the bloodstream is controlled primarily by the patch and not by the skin. The invention provides for a much smaller flux out of the patch than the skin can absorb, i.e., the patch controls the delivery of the drug to the skin and the skin has little impact on the permeation resistance.

J_{T} is given by (J_{P}×J_{S})/(J_{P}+J_{S}) where J_{P} is the flux of drug from the transdermal system or “patch” (into water) and J_{S} is the flux of drug across the skin, where flux, it will be understood, is the release rate (from the system) or transport rate (through the skin) per unit area. The following examples clarify the concepts.

$${J}_{P} = 10 {J}_{S} →︀ {J}_{T} = ( 10 {J}_{S} \times {J}_{S} ) / ( 11 {J}_{S} ) = 10 / 11 {J}_{S} ∼ {J}_{S} ( skin - controlled release ) \qquad ( 1 )$$

$${J}_{S} = 10 {J}_{P} →︀ {J}_{T} = ( 10 {J}_{P} \times {J}_{P} ) / ( 11 C ) = 10 / 11 {J}_{P} ∼ {J}_{P} ( system - controlled release ) \qquad ( 2 )$$

Eq 2 shows that J_{T} is approximately J_{P}, if J_{S} is, for example, 10 times larger than J_{P}. And vice versa, the total input rate into the bloodstream is determined by the skin, if the flux from the system is much larger than the skin can absorb. Eq. 1 shows that J_{T}˜J_{S}, when J_{P} is much larger (e.g., 10×) than J_{S}. The preferred patch-controlled delivery is accomplished by (a) increasing the skin's permeability and (b) utilizing polymeric barriers to control the delivery flux from the system to the skin. This is, in turn, achieved by selecting a barrier or controlling membrane of a liquid reservoir system that is heat-sealable to the backing material and has the preferred permeability for the enhancer and/or drug. For example, if the solvent-enhancer flux determines the drug flux thru the skin, then to construct a liquid-reservoir system, an ethylene-vinyl acetate membrane can be chosen with appropriate vinyl acetate content and thickness. For example, ALZA/Ciba Geigy's Estraderm® uses this ethanol flux control to adjust the estradiol flux into the body. If the membrane has the desired permeability for ethanol but possesses too large a permeation resistance for the drug, then the drug can be incorporated into the adhesive layer. The adhesive layer can be chosen to provide no or little resistance for drug transport and/or the solvent enhancer. If the adhesive layer provides resistance for the solvent flux the membrane characteristics have to be adjusted to provide less permeation resistance. The adhesive layer can also include plasticizer-type skin permeation enhancers that may not be able to penetrate the membrane at the desired rate. Those plasticizer-type skin permeation enhancers have a dual function as they can be used to adjust the pressure-sensitive adhesive in its adhesive and cohesive properties.

The transdermal systems of the invention include formulations wherein the active agent is contained in a “therapeutically effective” amount, i.e., in an amount effective to achieve its intended purpose. This means that, during the drug delivery period, the active agent is released from the transdermal system, into and through the skin, and ultimately into the bloodstream, to provide a therapeutically effective blood level of the active agent and/or an active metabolite thereof. The preferred mode of administration is via microdose, as alluded to above, where small amounts of active agent are periodically or, preferably and more typically, continuously released from the transdermal system into the skin at a predetermined flux that correlates with the desired pharmacological effect. This is in contrast to a “macrodose,” as is normally administered when an individual takes a psychedelic agent orally or in some other manner that results in substantially immediate release of an entire dose into the individual's bloodstream. Determination of a therapeutically effective amount for any particular active agent is within the capability of those skilled in the art.

The goal is to achieve desired brain activities ratings that are associated with the desired therapeutic effect. This follows from the Carhart-Harris Entropic Brain hypothesis; see Carhart-Harris (2018), supra. Carhart-Harris postulates that our brain states can be indexed by the magnitude of specific measures of brain entropy as determined by, for example, fMRI, EEC or MEG. In our normal, conscious state of mind, Carhart-Harris indicate that brain entropy falls within a certain range, termed a “critical zone,” that corresponds to being more or less awake and aware, with entropy or complexity of brain activity being neither too ordered nor too disordered. If one is asleep, sedated, or anesthetized, entropic brain activity is reduced beneath the critical zone. However, if one takes a standard “macrodose” of a psychedelic agent, entropy is shifted upward, above the critical zone, toward greater “conscious content, flexibility of mind and emotional lability” but analytical and convergent thinking are compromised. Administration of a psychedelic agent according to the invention avoids the upper region of the supercritical zone, although brain activity is heightened within the critical zone or slightly above it by continuous microdosing.

The intensity of psychedelic effects has been quantified; see Madsen et al. (2019), “Psychedelic Effects of Psilocybin Correlate with Serotonin 2A Receptor Occupancy and Plasma Psilocin Levels,” Neuropsychopharmacology 44 (7): 1328-1334. Madsen et al. determined the relationships between subjective intensity of the psychedelic experience (evaluated using a Likert scale), neocortical 5-HT_{2A}-R %-occupancy (percent change in cerebral 5-HT_{2A}-R binding relative to baseline, as measured by positron emission tomography scans), and plasma psilocin level, following oral administration of psilocybin to human subjects. The researchers found that 5-HT_{2A}-R %-occupancy is a key determinant for the psychedelic experience, and, furthermore, that plasma psilocin concentration is correlated with neocortical 5-HT_{2A}-R %-occupancy (see Madsen et al., FIG. 3). The invention enables adjustment of a subject's 5-HT_{2A}-R %-occupancy level into an optimal range, as will be described in detail infra.

### 3. The Active Agents

The psychedelic agents that are transdermally administered using the present invention are generally serotonergic psychedelic agents (serotonin 5-HT_{2A} receptor agonists) or empathogenic psychedelic agents (serotonin releasing agents).

Examples of serotonergic psychedelic agents include, without limitation, the following:
alkylated tryptamines such as psilocin, bufotenine, N,N-dimethoxytryptamine (DMT), baeocystin, aeruginascin, 5-methoxy-DMT, 5-bromo-DMT, N-methyl-N-ethyltryptamine (NET), N-methyl-N-isopropyltryptamine (MiPT), N-methyl-N-propyltryptamine (MPT), N,N-diethyltryptamine (DET), N-ethyl-N-isopropyltryptamine (EiPT), N-methyl-N-butyltryptamine (NBT), N-propyl-N-isopropyltryptamine, N,N-dipropyltryptamine, N,N-diisopropyltryptamine, N,N-diallyltryptamine, N,N-dibutyltryptamine, N-ethyltryptamine, N-methyltryptamine, trimethyltryptamine, α-methyltryptamine, α-ethyltryptamine, α, N-DMT, α,N,N-trimethyltryptamine, ethocybin, metocin, ethocin, meprocin, miprocin, deprocin, iprocin, daltocin, lucigenol, psilomethoxin, psilacetin (O-acetyl psilocin), metacetin, ethacetin, ipracetin, and derivatives thereof;
benzofurans such as dimemebfe and 5-methoxy-N, N-diisopropylbenzofuranethylamine;
ibogoids such as ibogaine and voacangine;
lysergamides such as lysergic acid diethylamide (LSD), lysergic acid amide, N1-methyl-lysergic acid diethylamide, N-acetyl-lysergic acid diethylamide, lysergic acid ethylamide, lysergic acid methyl ester, lysergic acid 2,4-dimethylazetitide; lysergic acid piperidine, N,N-dimethyl-lysergamide, lysergic acid β-propanolamide, lysergic acid 1-butanolamide, and other lysergic acid and lysergamide derivatives and analogs; and
phenethylamines such as mescaline, lophophine, isomescaline, cyclopropylmescaline, escaline, trisescaline, isoproscaline, methallylescaline, buscaline, 3-methoxy-4-ethoxyphenethylamine (MEPEA), β-methoxymescaline, and 2,5-dimethoxy, 4-substituted phenethylamines.

Another group of psychedelic active agents transdermally administrable according to the invention are the empathogens, i.e., serotonin releasing agents, including:
substituted amphetamines and substituted methylenedioxy-phenethylamines such as 3,4-methylenedioxymethamphetamine (MDMA), 3,4-methylenedioxyamphetamine (MDA), 2,3-MDA, 5-methyl-MDA, 3-methoxy-MDA), 3,4-methylenedioxy-N-ethylamphetamine (MDEA), 3,4-methylenedioxy-N-hydroxyamphetamine (MDOH), 3,4-ethylenedioxy-N-methylamphetamine (EDMA), p-methoxyamphetamine, and p-methoxymethamphetamine; and
substituted cathinones such as methylone, ethylone, eutylone, and pentylone.

An additional group of psychedelic agents that can be administered using the present invention are N-methyl-D-aspartate receptor antagonists, particularly ketamine.

In another embodiment, the systems, formulations, and methods of the invention deliver a psychedelic indolealkylamine transdermally, where the term “indolealkylamine” refers to a psychedelic indolealkylamine per se as well as to psychedelic analogs or derivatives thereof. The psychedelic indolealkylamines are analogues of 5-hydroxytryptamine (5-HT or serotonin), a monoamine neurotransmitter well known for its capability to affect mood and behavior. Examples of indolealkylamines suitable for use herein are psychedelic active agents having the molecular structure of formula (I)

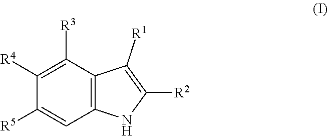

wherein:
R¹ is selected from amino, mono-substituted amino, di-substituted amino, amido, -L-R⁶ in which L is optionally substituted, optionally heteroatom-containing —(CH₂)ₙ— where n is an integer in the range of 1 to 6 inclusive and R⁶ is amino, mono-substituted amino, di-substituted amino, or a 5- or 6-membered, alicyclic or aromatic N-heterocycle, or wherein R¹ and R² taken together form a cyclic group containing at least one nitrogen atom;
R² is H or taken together with R¹ forms a cyclic group; and
R³, R⁴ and R⁵ are independently selected from H and OR⁷ wherein R⁷ is H, C₁-C₆ hydrocarbyl, or phosphonato, or wherein R³ and R⁴, or R⁴ and R⁵, taken together form a cyclic group.

More typically:
R¹ has the structure —(CH₂)ₙ—R⁶ where n is in an integer in the range of 2 to 4 inclusive and R⁶ is selected from amino, mono(alkyl)-substituted amino, and di(alkyl)-substituted amino;
R² is H;
R³ and R⁴ are independently selected from H and OH; and
R⁵ is selected from H and OR⁸ wherein R⁸ is H or lower alkyl.

In one embodiment, R² and R⁵ are both H and R⁶ has the structure —N(R⁹R¹⁰) in which R⁹ and R¹⁰ are independently selected from H and lower alkyl, such that the compound then has the structure of formula (II)

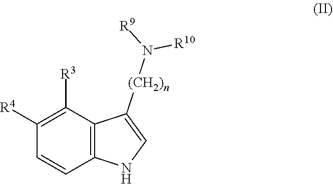

In one group of such compounds, R⁴ is H and the indolealkylamine of formula (II) has the structure of formula (III)

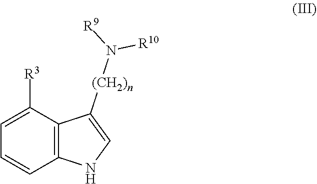

wherein R³ is H or OH; n is an integer in the range of 2 to 4 inclusive, and R⁹ and R¹⁰ are as defined previously, i.e., independently selected from H and lower alkyl. It will be appreciated that
when n is 2, and R⁹ and R¹⁰ are methyl, the indolealkylamine of Formula (III) is psilocin when R³ is OH and N, N-dimethyltryptamine (DMT) when R³ is H.

When R³ and R⁵ are H, the indolealkylamine has the structure of formula (IV)

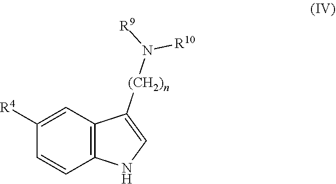

wherein R⁴ is selected from H and OH, and n, R⁹ and R¹⁰ are as defined for formulae (II) and (III). The indolealkylamine of Formula (II) is bufotenine, a structural isomer of psilocin, when R⁴ is hydroxyl, n is 2, and R⁹ and R¹⁰ are methyl.

The psychedelic indolealkylamine may be administered as a monotherapy or it may be administered in combination with one or more other active agents.

### Additional Active Agents:

Any additional active agent(s) co-administered with the psychedelic agent may serve the same purpose as the psychedelic agent, or it may serve a different purpose. In some cases, synergy can be seen with respect to therapeutic efficacy. In some cases, synergy can be seen with respect to a reduction in side effects observed with any one of the active agents being co-administered. In some cases, the synergy allows for a reduction in the dosage of one or more active agents being co-administered.

With combination therapy, the additional active agent(s) may be incorporated into the same drug delivery system and co-administered simultaneously, or the additional active agent(s) may be administered using a separate transdermal system or via an alternative route, e.g., orally, intravenously, or the like. Co-administration of two or more active agents via “combination therapy” may thus be carried out simultaneously, or the two or more active agents may be administered sequentially, at different times.

Additional active agents that may be administered in combination with the psychedelic agent include analgesic agents; anesthetic agents; antiarthritic agents; anticancer agents, including antineoplastic drugs; anticonvulsants; antidepressants; antidiabetic agents; antidiarrheals; antihistamines; antihyperlipidemic agents; antihypertensive agents; anti-infective agents such as antibiotics and antiviral agents; anti-inflammatory agents; antimigraine preparations; antinauseants; antineoplastic agents; antioxidants; antiparkinsonism drugs; antipyretics; antispasmodics; anxiolytics; herbal remedies; hormonolytics; muscle relaxants; steroids; and tranquilizers. Typically preferred additional active agents are selected from anti-inflammatory agents, antiviral agents, anti-cancer agents, anti-apoptotic agents, neuroprotective agents, and antioxidants.

Specific pharmacologically active agent classes of interest that may be administered with the psychedelic agent include, by way of example and not limitation, those set forth below.

Monoamine oxidase (MAO) inhibitors represent an additional class of active agents that are advantageously co-administered with the psychedelic agent. MAO inhibitors are of particular interest with respect to psychedelic agents that are metabolized by MAO, e.g., psilocybin and bufotenine. Examples of MAO inhibitors include nonselective MAO-A/MAO-B inhibitors such as isocarboxazid, hydracarbazine, phenelzine, and tranylcypromine; selective MAO-A inhibitors such a bifemelane, moclobemnide, pirlindole, and toloxatone; reversible MAO-A inhibitors such as harmine, harmaline, and d-1,2,3,4-tetrahydroharmine; and selective MAO-B inhibitors such as rasagiline, selegiline, and safinamide. Any MAO inhibitor should be incorporated in a drug reservoir or separate formulation along with the MAO-reactive active agent, typically at a concentration in the range of about 0.5 wt. % to about 15 wt. %, e.g., 1.0 wt. % to about 10 wt. %.

Cannabinoids: The term “cannabinoid” refers to a chemical compound that is found in the Cannabis genus of the Cannabaceae plant family, which includes the species Cannabis sativa, Cannabis indica, and Cannabis ruderalis, or to a metabolite, derivative, or analogue thereof, whether naturally occurring or chemically synthesized. Cannabinoids have been established as having any of a wide range of pharmacological utility and are encompassed by more than one of the aforementioned categories.

Examples of cannabinoids that can be transdermally administered in combination with the psychedelic agent using the present systems, formulations, and methods include, without limitation, tetrahydrocannabinol (THC), dronabinol (i.e., the pure isomer (−)-trans-Δ⁹-THC), cannabichromanone, cannabichromene (CBC), cannabichromenic acid, cannabichromevarin (CBCV), cannabichromevarinic acid, cannabicitran (CBT), cannabicoumaronone (CBCON), cannabicyclol (CBL), cannabicyclolic acid, cannabicyclovarin, cannabidiol (CBD), cannabidiol monomethyl ether, cannabidiol dimethyl ether, cannabidiol dimethyl heptyl, cannabidiol dimethyl heptyl-7-oic acid, dimethyl heptylpentyl cannabidiol (DMHP-CBD), cannabidiolic acid, cannabidiorcol, cannabidivarin (CBV), cannabidivarinic acid, cannabielsoin (CBE), cannabielsoinic acid, cannabifuran, cannabigerol (CBG), cannabigerol monomethyl ether (CBGM), cannabigerolic acid, cannabigerolic acid monomethyl ether, cannabigerovarin (CBGV), cannabigerovarinic acid, cannabiglendol, cannabinodiol, cannabinodivarin, cannabinol (CBN), cannabinolic acid, cannabinol methyl ether, cannabiorcol, cannabiripsol, cannabitetrol, cannabitriol, 10-O-ethyl-cannabitriol, cannabivarichromene, cannabivarin, dehydrocannabifuran, 1,2-dihydroxyhexahydrocannabinol, 1,2-dihydroxyhexahydrocannabinol acetate, dimethylheptylpyran, isotetrahydrocannabivarin, levonantradol, nabilone, rimonabant, Δ⁹-tetrahydrocannabinolic acid, Δ⁹-tetrahydrocannabiorcol, Δ⁹-tetrahydrocannabiorcolic acid, Δ⁹-tetrahydrocannabivarin, Δ⁹-tetrahydrocannabivarinic acid, 8,11-dihydroxy-Δ⁹-tetrahydrocannabinol, 8,9-dihydroxy-Δ^{6a,10a}-tetrahydrocannabinol, Δ⁸-tetrahydrocannabinol, Δ⁸-isotetrahydrocannabinol, Δ⁸-tetrahydrocannabinolic acid, 10-OXO-Δ^{6a,10a}-tetrahydrocannabinol (OTHC), HU-210 (1,1-dimethylheptyl-11-hydroxy-Δ⁸-THC), HU-331 (3-hydroxy-2-[(1R)-6-isopropenyl-3-methyl-cyclohex-2-en-1-yl]-5-pentyl-1,4-benzoquinone), JWH-018 (1-pentyl-3-(1-naphthoyl) indole) and other JWH cannabinoids such as JWH-073 (John W. Huffman, Clemson University, Clemson, SC), AM-2201 (1-(5-fluoropentyl)-3-(1-naphthoyl) indole) and other AM cannabinoids (Alexandros Makriyannis, Northeastern University, Boston MA), and CP-55,940 (2-((1S,2S,5S)-5-hydroxy-2-(3-hydroxypropyl)cyclohexyl)-5-(2-methyloctan-2-yl) phenol; Pfizer). As analgesic agents, these compounds have been proposed for use in treating neuropathic or chronic pain associated with fibromyalgia, rheumatoid arthritis, acute inflammation, and cancer. See, e.g., A. Hazekamp (2010), “Review on Clinical Studies with Cannabis and Cannabinoids,” Cannabinoids 5 (special issue): 1-21. Other cannabinoids that can be administered in combination with the psychedelic agent will be apparent to those of ordinary skill in the art with reference to the pertinent texts, journals, and patent literature. See, e.g., U.S. Patent Publication No. 2014/0271940 Δ¹ to Wurzer et al., incorporated by reference herein.

Anti-inflammatory agents: These include nonsteroidal anti-inflammatory agents (NSAIDs) such as ketoprofen, flurbiprofen, ibuprofen, naproxen, fenoprofen, benoxaprofen, indoprofen, pirprofen, carprofen, oxaprozin, pranoprofen, suprofen, alminoprofen, butibufen, fenbufen, apazone, diclofenac, difenpiramide, diflunisal, etodolac, indomethacin, ketorolac, meclofenamate, nabumetone, phenylbutazone, piroxicam, sulindac and tolmetin; and steroidal anti-inflammatory agents, e.g., hydrocortisone, hydrocortisone-21-monoesters (e.g. hydrocortisone-21-acetate, hydrocortisone-21-butyrate, hydrocortisone-21-propionate, hydrocortisone-21-valerate), hydrocortisone-17,21-diesters (e.g. hydrocortisone-17,21-diacetate, hydrocortisone-17-acetate-21-butyrate, hydrocortisone-17,21-dibutyrate), alclometasone, dexamethasone, flumethasone, prednisolone and methylprednisolone.

Antioxidants: Antioxidants useful herein are bioactive antioxidants that are preferably, although not necessarily, naturally occurring compounds, chemically synthesized naturally occurring compounds, modified naturally occurring compounds, and/or compounds that have been approved by the FDA or other regulatory agency as safe to ingest. Antioxidants include, by way of example and not limitation, ascorbic acid, vitamin E (tocopherols and tocotrienols), carotenoids (e.g., beta-carotene, lycopene, lutein, and zeaxanthin), ubiquinol (coenzyme Q), glutathione, uric acid, and lipoic acid, as well as the synthetic antioxidants propyl gallate, tertiary butylhydroquinone, butylated hydroxyanisole (BHA), and butylated hydroxytoluene (BHT). For other antioxidants that can be delivered with the psychedelic agent, see Kebede et al. (2019), “Application of Antioxidants in the Food Processing Industry: Options to Improve the Extraction Yields and Market Value of Natural Products,” Adv. Food Tech. Nutr. Sci. Open J. 5 (2): 38-49, the disclosure of which is incorporated by reference herein.

Another group of active agents that can be co-administered with the psychedelic agent are the chemical compounds found in the Antrodia camphorata mushroom. The constituents of Antrodia camphorata include terpenoids, benzenoids, lignans, benzoquinone derivatives, and other compounds. Specific and representative Antrodia camphorata compounds useful herein are as follows:
terpenes and terpenoids such as humulene (α-caryophyllene), myrcene, pinene (including α-pinene and β-pinene), antrocin, 19-hydroxylabda-8(17)-en-16,15-olide, 3β, 19-dihydroxylabda-8(17), 11E-dien-16,15-olide, 13-epi-3β, 19-dihydroxylabda-8(17), 11E-dien-16,15-olide, 19-hydroxylabda-8 (17)-13-dien-16,15-olide, 14-deoxy-11,12-didehydroandrographolide, 14-deoxyandrographolide, pinusolidic acid, antcin A, antcin B (zhankuic acid A), antcin C, antcin D (zhankuic acid F), antcin E, antcin F, antcin G, antcin H (zhankuic acid C), antcin I (zhankuic acid B), antcin K, methyl antcinate A, methyl antcinate B, zhankuic acid D, methyl antcinate G, methyl antcinate H, zhankuic acid E, dehydroeburicoic acid, dehydrosulphurenic acid, 15-acetyl-dehydrosulphurenic acid, eburicoic acid, sulphurenic acid, versisponic acid D, eburicol (24-methylenedihydrolanosterol), 3β, 15α-dihydroxy-lanosta-7,9 (11),24-triene-21-oic acid, 3β-hydroxy-lanosta-7,9 (11),24-triene-21-oic acid, β-sitosterol, β-sitostenone, stigmasterol, ergosterol, ergosta-4,6,8 (14) 22-tetraen-3-on3, and epi-Friedelinol;
benzenoids, such as 1,4-dimethoxy-2,3-methylenedioxy-5-methylbenzene, 1,4-dimethoxy-2,3-methylenedioxy-5-benzoate, 1,6-dimethoxy-2,3-methylenedioxy-4-benzoic acid, antrocamphin A, antrocamphin B, 2,3,4,5-tetramethoxybenzoyl chloride, antrodioxolanone, and isobutylphenol;
lignans, such as (+)-sesamin, 4-hydroxy sesamin, and (−)-sesamin;
benzoquinone derivatives such as 5-methyl-benzo (1,3)-dioxole-4,7-dione, 2-methyoxy-5-methyl (1,4)benzoquinone, and 2,3-dimethoxy-5-methyl (1,4)benzoquinone,
succinic and maleic acid derivatives, such as trans-3-isobutyl-4-[4-(3-methyl-2-butenyloxy)phenyl]pyrrolidine-2,5-dione, trans-1-hydroxy-3-(4-hydoxyphenyl)-4-isobutylpyrrolidine-2,5-dione, 3R*,4S*-1-hydroxy-3-isobutyl-4-[4-(3-methyl-2-butenyloxy)phenyl]pyrrolidine-2,5-dione (antrodin D or camphorataimide E), cis-3-(4-hydroxyphenyl)-4-isobutyldihydrofuran-2,5-dione, 3-(4-hydroxyphenyl)-4-isolbutyl-1H-pyrrole-2,5-dione, 3-(4-hydroxypheryl)-4-isobutylfuran-2,5-dione (antrocinnamomin C), 3-isobutyl-4-[4-(3-methyl-2-butenyloxy)phenyl]furan-2,5-dione (antrodin A or camphorataanhydride A), dimethyl 2-(4-hydroxyphenyl)-3-isobutylmaleate, 3-isobutyl-4-[4-(3-methyl-2-butenyloxy)phenyl]-1H-pyrrole-2,5-dione (antrodin B or camphorataimide B), antrocinnamomin D, 3-isobutyl-4 [4-(3-methyl-2-nyloxy)phenyl]-1H-pyrrol-1-od-2,5-dione (antrodin C or) camphorataimide C), antrocinnamomins A, 3R*,4S*-1-hydroxy-3-isobutyl-4-[4-(3-methyl-2-butenyloxy)phenyl]pyrrolidine-2,5-dione (antrodin E or camphorataimide D), and antrocinnamomins B; and
miscellaneous compounds not encompassed within the following groups, including 2,2′,5,5′-tetramethoxy-3,4,3′,4′-bi-methylenedioxy-6,6′dimethylbiphenyl, α-tocospiro B, methyl oleate, antroquinonol, adenosine, cordycepin, 2,4,5-trimethoxybenzaldehyde, antroquinonol B, 4-acetyl-antroquinonol B, 2,3-(methylenedioxy)-6-methylbenzene-1,4-diol, and 2,4-dimethoxy-6-methylbenzene-1,3,diol.

See Geethangili et al. (2009), “Review of Pharmacological Effects of Antrodia camphorata and Its Bioactive Compounds,” Evidence-based Complementary and Alternative Medicine 2011:1-17.

In another embodiment, a constituent of the Antrodia camphorata mushroom as above is substituted for the psychedelic agent and administered as a monotherapy. These compounds have been identified as alternatives to current cancer therapies and treatment of immune-related diseases. Co-administration of the Antrodia camphorata constituent with a cannabinoid is also envisioned.

It should also be noted that the components of the Antrodia camphorata mushroom, particularly the terpenoids, serve as permeation enhancers of the lipid disrupting type and can be advantageously used as the plasticizer-type permeation enhancer herein.

Other additional active agents that can be co-administered with the psychedelic agent include, by way of example, the following:

Anti-microbial agents: Tetracycline antibiotics and related compounds (e.g. chlortetracycline, oxy-tetracycline, demeclocycline, methacycline, doxycycline, minocycline and roli-tetracycline); macrolide antibiotics such as erythromycin, clarithromycin, and azithromycin; streptogramin antibiotics such as quinupristin and dalfopristin; beta-lactam antibiotics, including penicillins (e.g., penicillin G, penicillin VK), antistaphylococcal penicillins (e.g. cloxacillin, dicloxacillin, nafcillin and oxacillin), extended spectrum penicillins (e.g. aminopenicillins such as ampicillin and amoxicillin, and antipseudomonal penicillins such as carbenicillin), cephalosporins (e.g. cefadroxil, cefepime, cephalexin, cefazolin, cefoxitin, cefotetan, cefuroxime, cefotaxime, ceftazidime and ceftriaxone) and carbapenems such as imipenem, meropenem and aztreonam; aminoglycoside antibiotics such as streptomycin, gentamicin, tobramycin, amikacin and neomycin; glycopeptide antibiotics such as teicoplanin; sulfonamide antibiotics such as sulfacetamide, sulfabenzamide, sulfadiazine, sulfadoxine, sulfamerazine, sulfamethazine, sulfamethizole and sulfamethoxazole; quinolone antibiotics such as ciprofloxacin, nalidixic acid and ofloxacin; anti-mycobacterials such as isoniazid, rifampin, rifabutin, ethambutol, pyrazinamide, ethionamide, aminosalicylic and cycloserine; systemic antifungal agents such as itraconazole, ketoconazole, fluconazole and amphotericin B; and miscellaneous antimicrobial agents such as chloramphenicol, spectinomycin, polymyxin B (colistin), bacitracin, nitrofurantoin, and methenamine.

Anti-convulsant agents: Azetazolamide, carbamazepine, clonazepam, clorazepate, ethosuximide, ethotoin, felbamate, lamotrigine, mephenyloin, mephobarbital, phenyloin, phenobarbital, primidone, trimethadione, vigabatrin, topiramate, and benzodiazepines.

Anxiolytics and tranquilizers: Benzodiazepines (e.g. alprazolam, brotizolam, chlordiazepoxide, clobazam, clonazepam, clorazepate, demoxepam, diazepam, estazolam, flumazenil, flurazepam, halazepam, lorazepam, midazolam, nitrazepam, nordazepam, oxazepam, prazepam, quazepam, temazepam and triazolam), buspirone, chlordiazepoxide and droperidol.

Anticancer and antineoplastic agents: Paclitaxel; docetaxel; camptothecin and its analogues and derivatives (e.g. 9-aminocamptothecin, 9-nitrocamptothecin, 10-hydroxycamptothecin, irinotecan, topotecan and 20-O-β-glucopyranosyl camptothecin); taxanes (e.g. baccatins, cephalomannine and their derivatives); carboplatin; cisplatin; interferon α-2a, interferon α-2b, interferon α-n3 and other agents of the interferon family; levamisole; altretamine; cladribine; tretinoin; procarbazine; dacarbazine; gemcitabine; mitotane; asparaginase; porfimer; amifostine; mitotic inhibitors including podophyllotoxin derivatives teniposide and etoposide; and the vinca-alkaloids vinorelbine, vincristine and vinblastine.

Anti-viral agents: These include anti-herpes agents such as acyclovir, famciclovir, foscarnet, ganciclovir, idoxuridine, sorivudine, trifluridine, valacyclovir and vidarabine; anti-retroviral agents such as didanosine, stavudine, zalcitabine, tenovovir and zidovudine; and other antiviral agents including amantadine, interferon-α, ribavirin and rimantadine.

Agents to treat neurodegenerative diseases: Active agents for treating Alzheimer's disease and Huntington's disease include donezepil, physostigmine, and tacrine, for treatment of Alzheimer's Disease, and fluoxetine and carbamazepine, for treating Huntington's Disease. Anti-Parkinsonism drugs useful herein include amantadine, apomorphine, bromocriptine, levodopa (particularly a levodopa/carbidopa combination), pergolide, ropinirole, selegiline, trihexyphenidyl, trihexyphenidyl hydrochloride, and anticholinergic agents. ALS is generally treated with spasmolytic (anti-spastic) agents such as baclofen, diazepam, tizanidine, and dantrolene.

Neuroprotective agents: glutamate blockers and NMDA channel blockers such as magnesium sulfate; free radical scavengers such as tempol, hydroxystilbene, and oxyreservatrol; COX-2 inhibitors such as flavocoxid, valdecoxib, and celecoxib; beta-blockers such as propranolol, metoprolol, and atenolol; statins such as atorvastatin, mevastatin, rosuvastatin, and simvastatin; melatonin; and erythropoietin.

Apoptosis inhibitors: These are compounds that inhibit a cell's initiation of or progression through the apoptosis process, and include inhibitors of c-Myc, Bax, p53, tBid, and BCL as well as caspase inhibitors and other enzymes involved in the apoptotic pathways. Examples of apoptosis inhibitors include the following: 5-[(4-ethylphenyl)methylene]-2-thioxo-4-thiazolidinone; 4′-methoxyflavone; 4-[2-methyl-1-(1-methylethyl)-1H-imidazol-5-yl]-N-[4-(methylsulfonyl)phenyl]-2-pyrimidinamine; 1H-benzimidazole-1-ethanol, 2,3-dihydro-2-imino-alpha-(phenoxymethyl)-3-(phenylmethyl)-monohydrochloride; N-[4-[(4-aminophenyl)thio]phenyl]-4-[(4-methoxyphenyl) sulfonyl]amino]-butanamide; 2-pyridin-2-yl-4H-1,3-benzothiazin-4-one; 4-chloro-3-[[(3-nitrophenyl)amino]sulfonyl]-benzoic acid; combretastatin A4; cyclic pifithrin-α-hydrobromide; fasentin; ferrostatin-1; 3-[6-[4-(trifluoromethoxy)phenyl]amino]-4-pyrimidinyl]benzamide (GNF-2); 2-(1H-Indol-3-yl)-3-pentylamino-maleimide (IM-54); ischemin; liproxstatin-1; Calpain Inhibitor III; N-ethylmaleimide; 4-chloro-2-[3-(3-trifluoromethyl-phenyl)-ureido]benzoic acid; necrostatin-1; and 3-(5-fluoro-1H-indol-3-yl)-2-mercapto-2-propenoic acid.

### 4. Drug Delivery Systems

A psychedelic active agent is delivered to a subject transdermally using a topically applied formulation or a transdermal drug delivery system as provided herein. As pointed out earlier herein, the desired concentration of the agent to be administered (or metabolite thereof) is controlled by the transdermal system insofar as the flux from the system dominates the total flux of active agent from the system through the skin and into the bloodstream. This feature of the invention facilitates the prolonged and controlled blood concentration levels, and “peaks” and “valleys” can be eliminated.

### (a) Topically Applied Formulation:

In one embodiment, the transdermal drug delivery system comprises an active agent-containing formulation to be applied to the skin, e.g., using a device for applying an intended (or “metered”) dose of a liquid formulation to the skin. For instance, a roll-on type of applicator such as those described in U.S. Pat. Nos. 8,419,307 and 9,289,586 to Bayly et al. can be used, as can a metered-dosage spray-on device.

The formulation comprises the psychedelic active agent, a skin permeation enhancer composition, and a pH-adjusting agent. The formulation is applied to a localized region of a subject's skin to administer a dose of active agent to be delivered over a drug delivery time period typically in the range of 6 to 84 hours, e.g., 8 to 24 hours. Upon evaporation or absorption of the enhancer composition, the direct enhancement effect of the enhancer composition ceases. Then, transport of the agent remaining on the application area or already in the skin can be reactivated by re-applying the enhancer composition in the absence of additional active agent. This step continues to promote the delivery of the drug that is already in and on the skin.

The concentration of the active agent in the formulation is selected to provide a unit dose of the active agent in each application. The unit dose, in turn, is selected to provide a therapeutically effective blood level of the active agent or a metabolite thereof during the drug delivery time period.

Enhancer compositions used in this embodiment comprise a combination of a solvent-type skin permeation enhancer and a plasticizer-type skin permeation enhancer, also referred to as a lipid disrupting agent.

Solvent-type enhancers include, without limitation, C₂-C₆ alcohols such as ethanol, isopropanol, 1,2-butanediol, and propylene glycol; ethers such as diethylene glycol monoethyl ether (available commercially as Transcutol®) and diethylene glycol monomethyl ether; ketones such as acetone; esters such as ethyl acetate and ethyl formate; hydrocarbon solvents such as pentane; amides and other nitrogenous compounds such as urea, N,N-dimethylacetamide (DMA), dimethyl formamide (DMF), 2-pyrrolidone, 1-methyl-2-pyrrolidone, ethanolamine, diethanolamine, and triethanolamine; alkanones such as acetone; organic acids, particularly ascorbic acid, citric acid and succinic acid; and sulfoxides such as dimethylsulfoxide (DMSO) and decylmethylsulfoxide (C₁₀MSO). Such enhancers increase the solubility of the active agent in the formulation and in the skin; some, such as ethanol, penetrate the skin relatively quickly and create a solvent-drag effect carrying dissolved active agent with it into the skin.

Plasticizer-type enhancers include, for instance, surfactants such as sodium laurate, sodium lauryl sulfate, cetyltrimethyl-ammonium bromide, benzalkonium chloride, poloxamers (e.g., Pluronic 231, 182, 184), and polysorbates (e.g., Tween 20, 40, 60, 80); polyethylene glycol and esters thereof such as polyethylene glycol monolaurate (PEGML); fatty alcohols, fatty acids, and esters of fatty acids such as oleic acid (OL), oleyl alcohol (OA), isopropyl myristate (IPM), methyl laurate, ethyl laurate, propylene glycol monolaurate, propylene glycol dilaurate, glycerol monolaurate, lauryl lactate, methyl laurate; glycerol phospholipids, including lecithin; higher order alcohols such as 2-phenoxyethanol (PHE), undecanol, hexanol, octanol, and benzoyl alcohol, and salicylaldehyde (SA); terpenes and terpenoids such as isomenthone, humulene, and other terpenes and terpenoids such as those identified by Kang et al. (2013), “Terpenes and Improvement of Transdermal Drug Delivery” in Ramawat, eds., Natural Products (Springer, Berlin); vegetable oils such as safflower oil, cotton seed oil, corn oil, almond oil, castor oil, jojoba oil, linseed oil, etc.; mineral oils and components thereof; and other solvents such as isomenthone, an ether/ester combination as described in U.S. Pat. No. 5,053,227, and 1-dodecylazacycloheptan-2-one (Azone®)).

The formulation additionally comprises a pH-adjusting agent, which may be, for example, a buffer system. Suitable buffer systems include phosphate buffers, bicarbonate buffers, and the like. The pH-adjusting agent may also be a weak acid such as ascorbic acid, citric acid, formic acid, acetic acid, or the like, depending on the active agent. The purpose of the pH-adjusting agent is to maintain the pH in the localized region of the active agent so that it approximates the pKa of the agent, generally bringing the pH to within about 25% of the pKa, typically within about 15% of the pKa. For psilocin, having a pKa of 8.47, a phosphate buffer is suitable, particularly a pyrophosphate buffer, as is sodium bicarbonate. A pyrophosphate buffer is also a suitable pH-adjusting agent for LSD, having a pKa of 7.8, and for DMT, having a pKa of 8.4. With bufotenine, having a pKa of 9.67, a phosphate buffer can also be used, but a buffer that provides a slightly higher pH might be preferable.

By approximating the active agent pKa, the pH-adjusting agent increases the concentration of the non-ionized form of a polar or ionizable active agent that is present and in equilibrium with the ionized form. When the formulation comprises an aqueous liquid carrier in addition to the aforementioned components, the pH-adjusting agent maintains the pH of the formulation at approximately the pKa of the active agent. In the absence of a liquid carrier, and upon delivery into the skin, it will be appreciated that the pH-adjusting agent maintains the pH in the region of the active agent to favor the non-ionized form of the drug, i.e., within the various layers of the skin.

The formulation may additionally comprise a stabilizing agent in order to stabilize the active agent, thereby preventing degradation or reaction of the active agent during storage and use. For example, some compounds, such as psilocin, undergo unwanted reactions in the presence of oxygen, as the phenolic hydroxyl group renders the molecule unstable and susceptible to radicalization and oligomerization; see, e.g., Lenz et al. (2020), “Injury-Triggered Blueing Reactions of Psilocybe “Magic” Mushrooms,” Angew Chem Int Ed Engl 59 (4): 1450-1454. Accordingly, a suitable stabilizing agent for psilocin and other oxygen-reactive active agents is an antioxidant. Antioxidants that may be advantageously incorporated into the formulation in such a case include those identified in the preceding section. A preferred antioxidant is one that serves an additional purpose in the formulation, reducing formulation complexity. Ascorbic acid and ascorbate salts are examples of suitable oxidants, insofar as they act i not only as an oxygen scavenger but also as a pH-adjusting agent and a solvent-type skin permeation enhancer.

The formulation typically comprises about 0.5 wt. % to about 40 wt. % active agent, more typically about 1 wt. % to about 20 wt. %, e.g., about 1 wt. % to about 10 wt. %, while the permeation enhancer composition typically represents about 1 wt. % to about 30 wt. % of the formulation, e.g., 1 wt. % to about 25 wt. %, such as 5 wt. % to about 25 wt. %, 1 etc. A liquid vehicle in addition to the enhancer composition may also be included. Depending on the active agent to be delivered and the components of the selected enhancer composition, the liquid vehicle may be hydrophilic or hydrophobic.

The formulation can also include one or more conventional additives, i.e., excipients, such as viscosity adjusting agents (i.e., thickening agents or thinning agents), crystallization inhibitors, emulsifiers, solubilizers, preservatives, opacifiers, colorants, fragrance, and the like. Furthermore, the pharmaceutical formulation may contain an irritation-mitigating additive such as glycerin to minimize or eliminate the possibility of skin irritation or skin damage that might result from a particular active agent or other component of the system.

In one embodiment, a biocompatible polymer is incorporated into the formulation that dries to become a film on the skin to create occlusivity and thereby increase skin hydration. Such polymers include poloxamers (e.g., Pluronic® block copolymers, available from BASF Corporation), acrylic acid polymers and copolymers (e.g., poly(acrylic acids such as those available from Lubrizol Corporation under the tradename Carbopol®), cosmetically acceptable film-forming polymers, including, without limitation: sulfopolyesters such as those available commercially as Eastman AQ™ polymers; silicone resins and silicone resin gums such as trimethylsiloxysilicate optionally admixed with cyclopentasiloxane, dimethicone, and polypropylsilsesquioxane, available as DOWSIL™ resins and resin gums from Dow Chemical Co.; silicone acrylates; ethylcellulose polymers; hydrophobic carboxylated acrylic copolymers; starches and modified starches (e.g., STRUCTURE® XL, from Nouryon); and others.

In another embodiment, the formulation includes one or more inhibitors of any enzymes that may cause unwanted or premature degradation of a specific active agent (e.g., MAO inhibitors for drugs such as psilocybin, psilocin, and bufotenine that are metabolized by MAO). Suitable MAO inhibitors are exemplified in the preceding section.

In a variation on this embodiment, the active agent or a fraction thereof is encapsulated within liposomes or ethosomes, i.e., liposomes containing a certain amount of ethanol. See Nounou et al. (2008), “Liposomal Formulation for Dermal and Transdermal Drug Delivery: Past, Present, and Future,” Recent Patents on Drug Delivery & Formulation 2:9-18; Pilch et al. (2018), “Liposomes with an Ethanol Fraction as an Application for Drug Delivery,” Int. J. Mol. Sci. 19:3806; and Somwanshi et al., “Development and Evaluation of Novel Ethosomal Vesicular Drug Delivery System of Sesamum indicum L. Seed Extract,” Asian J. Pharmaceut. 12 (4). The disclosure of the latter two references are incorporated by reference herein for their disclosures ethosomes that encapsulate drugs and ethosomal dispersions, respectively. Proliposomes, niosomes, proniosomes, transfersomes, and protransfersomes may be used in an analogous manner, although ethosomes are generally preferred. See Modi et al. (2012), “Transfersomes: New Dominants for Transdermal Drug Delivery,” Am. J. PharmTech Res. 2 (3). By incorporating a fraction of the total psychedelic agent into ethosomes or the like, e.g., 10 wt. % to 90 wt. %, 10 wt. % to 85 wt. %, or 20 wt. % to 75 wt. %, sustained release of the active agent after entry into the skin is prolonged beyond what would otherwise be the end of the drug delivery period, and the greater the fraction of the active agent that is incorporated into ethosomes, lithosomes, or the like, the longer the drug delivery period will be.

When the active agent administered is psilocin, the light sensitivity of the drug is offset by covering the application area with an opaque or metallized material after administration, which may be a film or layer that has a skin contact adhesive base layer, or on its edges or perimeter. A suitably opaque article of clothing may suffice. The potential chemical instability of the drug in the presence of oxygen is addressed by incorporation of an oxygen scavenger into the formulation, e.g., ascorbic acid, an enzyme or enzyme combination, a polyunsaturated fatty acid, or any of the antioxidants described as additional active agents in the preceding section.

In a variation on this embodiment, the active agent-containing formulation may also be applied to an area of a subject's skin using a metered dose transdermal spray. The formulation is composed of the same components as the roll-on formulation, unless an aerosolized device is used, in which case the components are incorporated into a volatile vehicle instead of the carrier alluded to above.

In one embodiment, the topically administrable formulation for transdermally administering a psychedelic active agent to a subject comprises:
(a) about 0.5 wt. % to about 40 wt. % of the psychedelic active agent, the active agent having a known pKa;
(b) an effective skin permeation-enhancing amount of an enhancer composition comprising a solvent-type enhancer and a lipid disrupting enhancer;
(c) an effective pH-adjusting agent amount of a pH-adjusting agent that maintains pH to within 25% of the pKa within a localized region of skin to which the formulation is topically applied; and, optionally,
(d) a stabilizing agent.

For instance, the formulation may comprise:
(a) about 1.0 wt. % to about 25 wt. % of the psychedelic active agent, the active agent having a known pKa;
(b) an effective skin permeation-enhancing amount of an enhancer composition comprising a solvent-type enhancer and a lipid disrupting enhancer;
(c) an effective pH-adjusting agent amount of a pH-adjusting agent that maintains pH to within 15% of the pKa within a localized region of skin to which the formulation is topically applied; and
(d) about 1.0 wt. % to about 10 wt. % of ascorbic acid or a salt thereof.

### (b) Transdermal Patches:

In another embodiment, an active agent-containing formulation comprising the active agent, the solvent-type enhancer/plasticizer-type enhancer combination, and the pH-adjusting agent, is incorporated into a drug reservoir within a laminated transdermal delivery system, or patch. The transdermal system includes a backing layer, preferably occlusive, and typically opaque or metallized, wherein the backing layer serves as the outer surface of the system following application of the patch to the skin of a subject. This type of backing layer prevents light from entering the patch and potentially destabilizing a light-sensitive drug such as psilocin. During storage and prior to use, the system also includes a removable release liner that covers and protects the skin-facing side of the adhesive matrix before use.

The backing layer is typically, although not necessarily, the primary structural element of the transdermal system following assembly of the laminate, and can be selected so as to provide the device with physical characteristics such as flexibility, drape, and, if desired, occlusivity. The material selected to serve as the backing layer should be stable under storage conditions, chemically inert with respect to any components of the adhesive layer laminated thereto, and incapable of absorbing formulations and formulation components contained within the transdermal drug delivery system. The backing is preferably made of one or more sheets or films of a flexible elastomeric material that serves as a protective covering to prevent loss of drug and/or vehicle via transmission through the upper surface of the assembled system. In addition, the backing material may be chosen to impart a degree of occlusivity to the device, such that the area of the skin covered on application becomes hydrated. The material used for the backing layer should permit the assembled transdermal system to follow the contours of the skin and be worn comfortably on areas of skin such as at joints or other points of flexure, areas that are normally subjected to mechanical strain, with little or no likelihood of the system disengaging from the skin due to differences between the flexibility or resiliency of the skin and the flexibility or resiliency of the system.

Examples of materials useful for the backing layer are polyesters, polyethylene, polypropylene, polyurethanes, polyether amides, and ethylene-vinyl acetate copolymers (EVA). Backing layers may be obtained commercially, for instance under the Scotchpak™ and CoTran™ brands from 3M Corporation, including polyester film backings (3M Scotchpak™ 9754, 9757, and 9758), polyester film laminate backings (3M Scotchpak™ 1012, 9723, 9730, 9733, 9735, and 9738), polyurethane nonwoven backings (3M CoTran™ 9700), polyurethane monolayer film backings (3M CoTran™ 9701), and polyethylene monolayer film backings (3M CoTran™ 9718, 9719, 9720, and 9722).

The backing layer is generally in the range of about 10 microns to about 300 microns in thickness, preferably in the range of about 15 microns to about 250 microns in thickness, and may, if desired, be pigmented, metallized, or provided with a matte finish suitable for writing.

### (i) Simple Adhesive System:

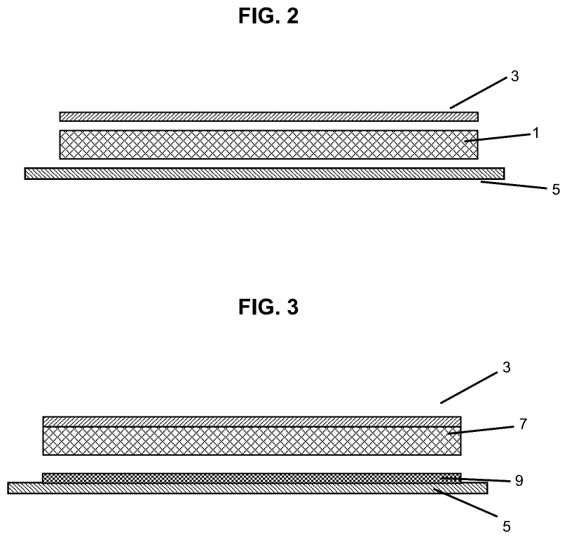

In one embodiment, the transdermal patch is a simple adhesive system, also referred to as a “drug-in-adhesive” system. The simple adhesive system is illustrated in FIG. 2, wherein drug reservoir 1 comprises a polymeric matrix that doubles as a skin contact adhesive (SCA) layer and serves to affix the system to the skin during drug delivery. The drug reservoir, comprising the components of the formulation described in the preceding section, is laminated directly to the outer backing layer 3.

Suitable skin contact adhesives are generally pressure-sensitive adhesives (PSAs), and preferably comprise a visco-elastic polymer, such as may be selected from polysiloxanes (silicones), polyisobutylenes (PIBs), polyacrylates, polyurethanes, and tacky rubbers other than PIB, such as polystyrene-isoprene copolymers, polystyrene-butadiene copolymers, and mixtures thereof. In addition to the elastomeric polymer, the PSA composition can also include a tackifying resin, a filler, a stabilizer and/or antioxidant, and a cross-linking agent, all selected to provide the desired degree of tack, peel adhesion, skin adhesion, and cohesive strength.

The skin-contacting surface of the SCA reservoir is protected during storage and prior to use by a removable release liner, shown in FIG. 2 as element 5. Release liners are typically composed of polyesters or other polymers that are treated with silicone coatings, fluorosilicone coatings, or coatings of other fluoropolymers. Commercially available release liners are available, and include, by way of example, Syl-Off® products (available from the Dow Corning Corporation), Tribex Corporation products, and 3M Scotchpak™ products. For an illustration of this type of transdermal drug delivery system, see Rastogi et al. (2012), “Transdermal Drug Delivery System: An Overview,” Asian J Pharmaceut. 6 (3): 161-170, at page 167, FIG. 3(c). The disclosure of Rastogi et al. is hereby incorporated by reference.

### (ii) Monolithic Matrix System:

In another embodiment, the transdermal drug delivery system used herein is a monolithic matrix system, in which the drug reservoir is again a polymeric matrix loaded with the active agent-containing formulation but in this case does not double as the means for affixing the system to the skin. The monolithic matrix system is illustrated in FIG. 3. The matrix reservoir 7 may or may not be comprised of a skin contact adhesive. Rather, a separate skin contacting adhesive layer 9 is laminated to the underside of the drug reservoir. Materials other than SCAs that are suitable as the matrix reservoir in this embodiment include, without limitation, polyurethanes, acrylic acid polymers and copolymers, poly(lactide-co-glycolide) (PLGA), and others, as will be known to those of ordinary skill in the art. Rastogi et al. (2012) illustrates a matrix system of this type on page 167, at FIG. 3(b). In FIG. 3, the preferably occlusive outer backing layer is shown at 3 and the release liner is shown at 5.

### (c) Liquid Reservoir Systems (LRS):

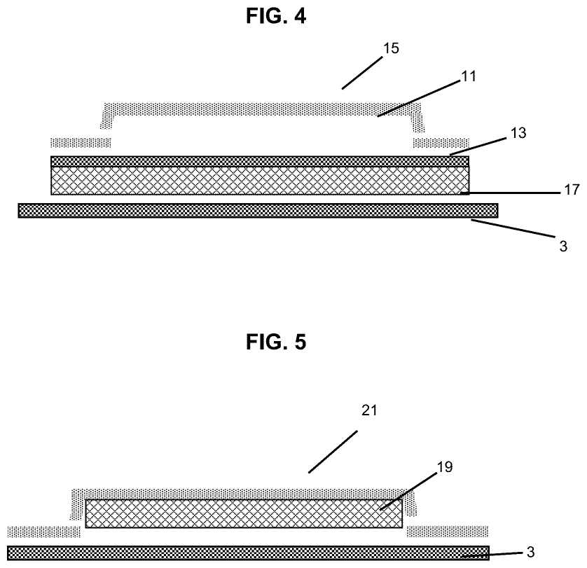

In these types of systems, the active agent-containing formulation as described in the preceding section is incorporated into a liquid drug reservoir instead of an adhesive matrix type of reservoir. In a first embodiment, termed LRS-m herein (m=membrane) and illustrated in FIG. 4, the transdermal system for drug administration has a reservoir 11 in the form of a pouch, or cavity, for housing the liquid formulation, where the reservoir is positioned between a heat-sealed lower membrane 13 and an occlusive outer backing layer 15. The heat-sealed membrane, which serves as a release “rate-controlling membrane,” is just inside a lower layer 17 comprising a skin contact adhesive that serves as the basal surface of the system and adheres to the skin of the subject during drug administration. In FIG. 4, the release liner is shown at 3. In this embodiment, the release rate of the enhancer composition and/or active agent can be controlled by the membrane so that delivery to the patient is controlled by the transdermal system and not by the skin. If the membrane permeability is designed to control the solvent-type enhancer flux, which in turn is correlated with the drug flux through the skin, then the drug can be contained within the adhesive layer as well as the drug reservoir. Suitable membrane materials include, by way of example, polyethylene, polypropylene, ethylene vinyl acetate copolymers, ethylene copolymers, ethylene oxide copolymers, polyamides, cellulosics, polyurethanes, polyether-blocked amide copolymers, and polyvinyl acetate. Some membranes may be microporous, e.g., those fabricated from microporous polyethylene or polypropylene.

In another embodiment, illustrated in FIG. 5, the active agent-containing formulation is incorporated into a microporous reservoir 19 laminated to an outer backing material 21, which, as before, is preferably occlusive and is opaque or metallized to prevent light from reaching a light-sensitive drug. This is termed the LRS-b (band aid) system. The microporous reservoir filled with the drug/enhancer formula is covered with a release liner 3 during storage and prior to use, as before. In this case, the LRS-b can be loaded with the active agent-containing formulation immediately prior to application to a subject's skin.

The LRS-m and LRS-b patches are illustrated in Rastogi et al. (2012) on page 167, at FIG. 3(a) (where the LRS-m system is referred to as a “reservoir system”) and FIG. 3(d) (where the LRS-b system is referred to as a “microreservoir system”).

### (d) Modular Transdermal Delivery Systems:

In another embodiment, a modular transdermal delivery system is used to deliver the active agent-containing formulation to a subject's skin. Suitable modular transdermal delivery systems are described in U.S. Patent Publication Nos. 2020/0268681 A1, 2020/0146998 A1, 2018/0311180 A1, 2018/0311181 A1, and 2018/0296498 A1, all to Frank Kochinke, the disclosures of which are incorporated by reference herein in their entireties. These modular systems are advantageous in clinical testing when different formulas are undergoing evaluation, insofar as they eliminate the need for a large scale manufacturing setup to produce clinical supplies. The modular patch components are pre-prepared and can be assembled and loaded by a researcher, manufacturer, or end user.

### 5. Methods of Use and Indications

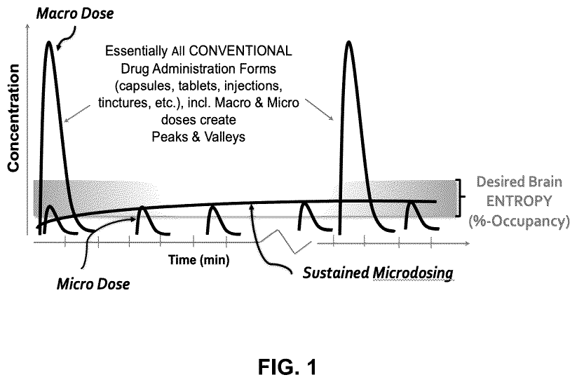

As explained earlier herein, a purpose of the invention is to administer a psychedelic active agent to a subject to achieve a desired therapeutic effect without resulting in unwanted adverse effects that would likely occur upon administration of a macrodose. See FIG. 1, which illustrates the plasma concentration profile obtained using the transdermal microdosing method of the invention compared with that obtained using conventional immediate release delivery, which involves administration of a drug macrodose.

In one embodiment, the invention provides a method for transdermally administering a psychedelic agent such as psilocin to a subject to increase 5-HT_{2A}-R %-occupancy to a level in the range of about 5% to about 60%, e.g., in the range of about 20% to about 50%. When the psychedelic agent is psilocin, the aforementioned 5-HT_{2A}-R %-occupancy range is achieved with a transdermal system that continuously delivers the active agent to provide and maintain a plasma psilocin concentration in the range of about 1.0 μg/L to about 7.5 μg/L, for instance 1.5 μg/L to about 5.0 μg/L.

The method is implemented in a method of use to treat a mental health condition in a subject. Examples of mental health conditions that can be treated herein include, without limitation, traumatic brain injury and stroke, mild traumatic brain injury (mTBI), opioid withdrawal, addiction, smoking cessation, Attention Deficit Hyperactivity Disorder (ADHD), dementia, Post Traumatic Stress Disorder (PTSD), Autism Spectrum Disorder (ASD), social phobias, obesity, anorexia bulimia, pain, mania, Generalized Anxiety Disorder (GAD), agoraphobia, specific phobias, panic disorder, separation anxiety disorder, acute stress disorder, adjustment disorders, reactive attachment disorder, somatic symptom disorder, illness anxiety disorder, anorexia nervosa, bulimia nervosa, rumination disorder, restless leg syndrome, obsessive-compulsive disorder, postpartum depression, schizophrenia, tic disorders including Tourette's syndrome, dysthymia (mild chronic depression), alcohol use disorder, season affective disorder, major depressive disorder (treatment-resistant depression), body dysmorphic disorder, depression in Alzheimer's Disease or dementia, dementia with Lewy Bodies, Parkinson's disease dementia, bipolar disorders 1 and 2, post-concussion headache, and depression in terminally ill patients.

In another embodiment, the invention is used to treat an inflammatory and/or auto-immune condition in a subject. Representative such conditions include neuropathy, sarcoma, Parkinson's disease, Parkinson's disease psychosis, cluster headaches, irritable bowel syndrome, chronic obstructive pulmonary disease, asthma, migraines, atherosclerosis, achalasia, Addison's disease, Adult Still's disease, agammaglobulinemia, alopecia areata, amyloidosis, ankylosing spondylitis, anti-GBM/anti-TBM nephritis, antiphospholipid syndrome, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune urticaria, axonal & neuronal neuropathy (AMAN), Baló disease, Bechet's disease, benign mucosal pemphigoid, bullous pemphigoid, Castleman disease (CD), celiac disease, Chagas disease, chronic inflammatory demyelinating polyneuropathy (CIDP), chronic recurrent multifocal osteomyelitis (CRMO), Churg-Strauss syndrome (CSS) or eosinophilic granulomatosis (EGPA), cicatricial pemphigoid, Cogan's syndrome, cold agglutinin disease, congenital heart block, coxsackie myocarditis, Crest syndrome, Crohn's disease, dermatitis herpetiformis, dermatomyositis, Devic's disease (neuromyelitis optica), discoid lupus, Dressler's syndrome, endometriosis, eosinophilic esophagitis (EOE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, glomerulonephritis, Goodpasture's syndrome, granulomatosis with polyangiitis, Graves' disease, Guillain-Barre syndrome, Hashimoto's thyroiditis, hemolytic anemia, Henoch-Schonlein purpura (HSP), herpes gestationis or pemphigoid gestationis (PG), hidradenitis suppurativa (HS) (acne inversa), hypogammaglobulinemia, IGA nephropathy, IGG4-related sclerosing disease, immune thrombocytopenia purpura (ITP), inclusion body myositis (IBM), interstitial cystitis (IC), juvenile arthritis, juvenile diabetes (type 1 diabetes), juvenile myositis (JM), Kawasaki disease, Lambert-Eaton syndrome, leukocytoclastic vasculitis, lichen planus, lichen sclerosus, ligneous conjunctivitis, linear IgA disease (LAD), lupus, Lyme disease chronic, Meniere's disease, microscopic polyangiitis (MPA), mixed connective tissue disease (MCTD), Mooren's ulcer, Mucha-Habermann disease, multifocal motor neuropathy (MMN) or MMNCB, multiple sclerosis, myasthenia gravis, myositis, narcolepsy, neonatal lupus, neuromyelitis optica, neutropenia, ocular cicatricial pemphigoid, optic neuritis, palindromic rheumatism (PR), PANDAS, paraneoplastic cerebellar degeneration (PCD), paroxysmal nocturnal hemoglobinuria (PNH), Parry Romberg syndrome, pars planitis (peripheral uveitis), Parsonage-Turner syndrome, pemphigus, peripheral neuropathy, perivenous encephalomyelitis, pernicious anemia (PA), POEMS syndrome, polyarteritis nodosa, polyglandular syndromes type I, II, III, polymyalgia rheumatica, polymyositis, postmyocardial infarction syndrome, post-pericardiotomy syndrome, primary biliary cirrhosis, primary sclerosing cholangitis, progesterone dermatitis, psoriasis, psoriatic arthritis, pure red cell aplasia (PRCA), pyoderma gangrenosum, Raynaud's phenomenon, reactive arthritis, reflex sympathetic dystrophy, relapsing polychondritis, restless legs syndrome (RLS), retroperitoneal fibrosis, rheumatic fever, rheumatoid arthritis, sarcoidosis, Schmidt syndrome, scleritis, scleroderma, Sjögren's syndrome, sperm & testicular autoimmunity, stiff person syndrome (SPS), subacute bacterial endocarditis (SBE), Susac's syndrome, sympathetic ophthalmia (SO), Takayasu's arteritis, temporal arteritis/giant cell arteritis, thrombocytopenia purpura (TTP), thyroid eye disease (TED), Tolosa-Hunt syndrome (THS), transverse myelitis, type 1 diabetes, ulcerative colitis (UC), undifferentiated connective tissue disease (UCTD), uveitis, vasculitis, vitiligo, and Vogt-Koyanagi-Harada Disease.

Specific indications of interest that can be treated using the systems, formulations, and methods of the invention include: neuropathic pain; post-traumatic stress disorder (PTSD); obesity; depression and anxiety, including end-of-life anxiety; trauma pain; neurodegenerative diseases such as Alzheimer's Disease; cluster headaches; and alcoholism.

### EXPERIMENTAL

### Example 1

### Psilocin Preparation

The following manufacturing method permits rapid isolation of psilocin from hallucinogenic mushrooms by coextraction of both psilocin and psilocybin. Dilute acetic acid is used as a solvent because both compounds are very soluble in acetic acid and very little interfering substances are extracted. Psilocybin is completely dephosphorylated to psilocin by heating the acid extract. After addition of a base, extraction into ether is performed promptly to avoid any possible decomposition of psilocin at a pH greater than 7. The method, extraction and dephosphorylation steps produce reasonably pure psilocin from a small amount of mushroom material.

### Protocol:

1. A representative sample of 2 to 10 g of dried mushrooms is ground to a fine powder by mortar and pestle.

2. The powder is mixed with 100 mL of dilute acetic acid in a 250-ml beaker. The pH is readjusted to pH 4 with glacial acetic acid.

3. After standing 1 h, the beaker is placed in a boiling water bath for 8 to 10 min or until the internal temperature of the acid mixture reaches 70 C.

4. The beaker is removed and cooled to room temperature under running water.

5. Then the acid mixture is separated from the mushroom powder by suction filtration using glass wool.

6. The filtrate is brought to pH8 with concentrated ammonium hydroxide and quickly extracted with two 50 mL portions of diethyl ether. Gentle mixing instead of shaking should be used to prevent an emulsion.

7. The ether is dried over sodium sulfate, filtered, and evaporated under nitrogen with no applied heat.

8. Crude psilocin will appear as a greenish residue. Recrystallization from chloroform/n-heptane (1:3) yields white crystals.

See Casale (1985), “An Aqueous-Organic Extraction Method for the Isolation and Identification of Psilocin from Hallucinogenic Mushrooms,” Journal of Forensic Sciences, JFSCA 30 (1): 247-250.

In order to ascertain the neocortical 5-HT_{2A} receptor occupancy level associated with the optimal therapeutic effect, tests are conducted with human volunteers in a psychedelic agent is transdermally administered to the volunteers using the transdermal systems, formulations, and methods of the invention. Plasma levels of the active agent or metabolite thereof are determined at regular time intervals, and correlated with each volunteer's subjective experience of the drug. A suitable method is a modification of the evaluation carried out and described by Madsen et al. (2018), wherein a transdermal system or formulation is substituted for oral drug administration. Optimal plasma level and the associated 5-HT_{2A} receptor occupancy level are determined, and a transdermal system is then fabricated to provide active agent release throughout a drug delivery time period so that the optimal plasma level is achieved and is approximately constant throughout drug delivery. This can be accomplished by using any or all of the following techniques: increasing or decreasing drug loading in the formulation/reservoir; increasing or decreasing the amount of enhancer; altering the ratio of enhancer to active agent; testing different enhancer compositions and pH-adjusting agents; incorporating various optional excipients such as viscosity adjusting agents, active agent stabilizers, and the like; or by using a rate-controlling membrane that increases or decreases release rate to a desired extent.

With psilocin, specifically, the Madsen et al. findings provide a starting point for identifying optimal plasma psilocin levels that correlate with a desired 5-HT_{2A} receptor occupancy levels. According to the inventors' testing, the skin flux of psilocin is in the range of 1 μg/cm²/hr to 10 g/cm²/hr, on average 5 μg/cm²/hr. With patch area of 50 cm² the dose per hour input ranges from 50 μg/hr/patch to 500 μg/hr/patch. At this patch size, the high end of the hourly input is approaching that of a 1-mg intravenous application. The daily dose ranges from 1.2 mg/day/patch to 12 mg/day/patch. At a skin flux of 5 μg/cm²/hr, the hourly input is 250 μg/hr/patch and the daily dose is 6 mg/day/patch. A suitable transdermal system for accomplishing the aforementioned delivery profile can be constructed as described above.

### Example 2

### Transdermal Psilocin System A

Psilocin (0.129 g) was added to 3.043 g ethanol. This ethanol/psilocin solution was then added to 3.034 grams of Duro-Tak® 87-4098 adhesive. The final mixture, in a vial, was put on a roller for 6 hours.

The solution was poured onto a 3M® Fluoropolymer Release Liner Film 9755. A casting knife, with a gap width of 330 μm, was used to evenly distribute the psilocin-containing adhesive. The cast solution was left to dry for 6 hrs. After drying, 3M Polyester Backing Film 1109 was placed on top of the dried solution. A patch with a surface area of 1 cm² was punched out and utilized for the in vitro diffusion test. The patch had a thickness of 130 μm and 4.12% loading of psilocin.

The cast adhesive solution, after drying, had an approximately 4 wt. % psilocin. It should be noted that the weight can vary, e.g., from 2 wt. % to about 15 wt. %. The Duro-Tak adhesive used was an acrylate copolymer-based adhesive, but other adhesives, particular acrylate adhesives, can also be used.

Skin from patient 1 (male, age 30) was used for the in vitro diffusion study in a Franz cell. A 1 cm² disk was punched out. The 1 cm² patch was placed on top of the 1 cm² skin disk; this was then placed in a membrane. Samples were collected on an hourly basis to test for psilocin diffusion. The temperature of the water used as the receptor medium was 32° C., and a pH of 7.4 was maintained using a PBS buffer. A flow rate of 0.12 mL/min was used. Two tests were run in parallel, designated A1 and A2.

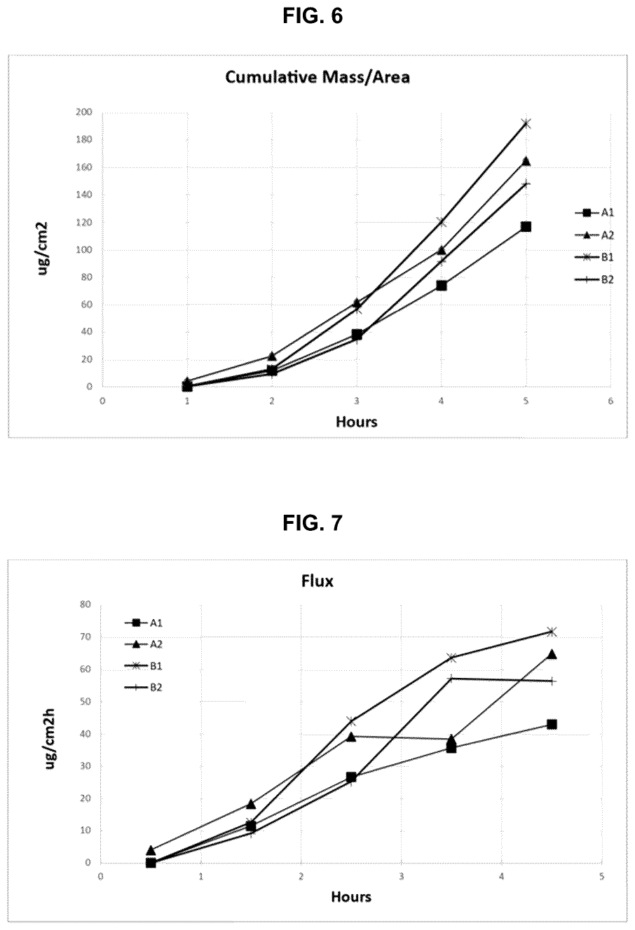

Cumulative mass released was plotted as a function of time for the first six hours, presented in FIG. 6 along with the results obtained for the transdermal system of Example 3, below. Skin flux over time was plotted and is shown in FIG. 7, along with the flux obtained for the transdermal system of Example 3.

### Example 3

### Transdermal Psilocin System B

The procedure of Example 2 was repeated, except that the patch had 3.89% psilocin loading. The same process was used to evaluate cumulative mass released and skin flux, with each test run twice and designated B1 and B2. The results obtained for transdermal psilocin tests A1, A2, B1, and B2 are shown FIGS. 6 and 7, respectively, as indicated above.

Each transdermal system exhibited a lag time of about two hours, as can be seen in FIG. 6. FIG. 7 shows that flux levels off around 70 μg/cm²/hr. From this data, one can deduce that a transdermal system made with the formulation parameters of Examples 2 and 3 and having a patch size of about 25 cm² will deliver around 42 mg of psilocin per day.

1. A method for administering psilocin to a subject in a controlled release manner, comprising:
applying a transdermal psilocin patch to a localized region of the subject's skin, the patch comprising a psilicin-containing drug reservoir, a backing layer that serves as the outer surface of the patch, and a skin-contacting basal surface comprising a skin contact adhesive;
allowing the transdermal psilocin patch to remain in place over an extended drug delivery period of about 6 hours to 84 hours,
wherein the transdermal psilocin patch provides a total input flux J_{T} of psilocin from the system, through the skin, and into the bloodstream equivalent to
$$( {J}_{P} \times {J}_{S} ) / ( {J}_{P} + {J}_{S} )$$
in which J_{P} is the flux of psilocin released from the transdermal patch and J_{S} is the flux of psilocin through the skin and into the bloodstream, and
further wherein the patch is configured to provide:
(a) a ratio of J_{S} to J_{P} in the range of about 2:1 to about 100:1, such that Jr is patch-controlled;
(b) continuous microdose delivery of the psilocin during the extended drug delivery period, said continuous microdose delivery effective to provide a plasma psilocin concentration in the subject in the range of about 1.0 μg/L to about 7.5 μg/L; and
(c) a 5-HT_{2A} occupancy level in the range of about 5% to about 60%.

2. The method of claim 1, wherein the continuous microdose delivery comprises providing a plasma psilocin concentration in the subject in the range of 1.5 μg/L to about 5.0 μg/L.

3. The method of claim 1, wherein the continuous microdose delivery comprises a psilocin dose in the range of 50 μg/hr/patch to 500 μg/hr/patch.

4. The method of claim 1, wherein the extended drug delivery period is in the range of about 8 hours to about 24 hours.

5. The method of claim 1, wherein the ratio of J_{S} to J_{P} is about 10:1.

6. The method of claim 1, wherein the drug reservoir houses a formulation comprised of about 0.5 wt. % to about 40 wt. % psilocin.

7. The method of claim 6, wherein the drug reservoir houses a formulation comprised of about 2.5 wt. % to about 25 wt. % psilocin.

8. The method of claim 7, wherein the formulation further comprises a monoamine oxidase (MAO) inhibitor in an amount effective to inhibit psilocin-degrading monoamine oxidases in the skin.

9. The method of claim 8, wherein the formulation further comprises a solvent.

10. The method of claim 9, wherein the formulation further includes a pH-adjusting agent effective to shift equilibrium of ionized psilocin and non-ionized psilocin at the localized region of the skin by increasing concentration of the non-ionized psilocin relative to the ionized psilocin.

11. The method of claim 10, wherein the pH-adjusting agent maintains pH within the localized region in the range of 7.20 to 9.74.

12. The method of claim 11, wherein the formulation further comprises a lipid disrupting enhancer.

13. The method of claim 1, wherein the patch provides a 5-HT_{2A} occupancy level in the range of about 20% to about 50%.

14. The method of claim 1, wherein the drug reservoir comprises a polymeric matrix.

15. The method of claim 14, wherein the polymeric matrix comprises the skin contact adhesive and provides the basal surface that affixes the patch to the skin.

16. The method of claim 1, wherein the drug reservoir comprises an enclosed pouch and the formulation is in liquid form.

17. The method of claim 1, wherein the drug reservoir houses a formulation comprised of:
2.5 wt. % to 25 wt. % psilocin;
1.0 wt. % to 15 wt. % of a monoamine oxidase inhibitor;
1.0 wt. % to 15 wt. % ascorbic acid;
a buffer that maintains pH within the localized region in the range of 7.20 to 9.74; and
an effective skin-permeation enhancing amount of an enhancer composition comprising a solvent enhancer and a lipid disrupting enhancer.

18. The method of claim 17, wherein buffer comprises a bicarbonate buffer or a phosphate buffer.

19. The method of claim 18, wherein:
the solvent enhancer is selected from C₂-C₆ alcohols, ethers, ketones, esters, hydrocarbon solvents, amides, urea, alkanones, organic acids, and sulfoxides; and
the lipid disrupting enhancer is selected from surfactants, polyethylene glycol, polyethylene glycol esters, fatty alcohols, fatty acids, fatty acid esters, higher order alcohols, terpenes, terpenoids, vegetable oils, mineral oils and components thereof, isomenthone, 1-dodecylazacycloheptan-2-one, and combinations thereof.

20. The method of claim 19, wherein the solvent enhancer is selected from ethanol, isopropanol, 1,2-butanediol, propylene glycol, diethylene glycol monoethyl ether, diethylene glycol monomethyl ether; acetone, ethyl acetate, ethyl formate, pentane, urea, N,N-dimethylacetamide, dimethyl formamide, 2-pyrrolidone, 1-methyl-2-pyrrolidone, ethanolamine, diethanolamine, triethanolamine, dimethylsulfoxide and decylmethylsulfoxide.
